(19) United States
(12) Patent Application Publication
(10) Pub. No.: US 2012/0108994 A1
(43) Pub. Date: May 3, 2012

(54) MORPHOLOGY CHANGE DETECTION FOR CARDIAC SIGNAL ANALYSIS

(76) Inventors: Amisha S. Patel, Maple Grove, MN (US); Jeffrey A. Hayden, Whitehall, MI (US)

(21) Appl. No.: 13/017,339

(22) Filed: Jan. 31, 2011

Related U.S. Application Data
(60) Provisional application No. 61/408,244, filed on Oct. 29, 2010.

(51) Int. Cl. A61B 5/0452

(52) U.S. Cl. 600/515

(57) ABSTRACT

Method and apparatus for improved detection of changes in morphology for cardiac analysis in post-processing. In some examples, a method of detecting a morphology change includes receiving an electrogram signal that represents a heartbeat; calculating a plurality of correlation values between the heartbeat and a template heartbeat; determining a maximum correlation value between the heartbeat and the template heartbeat based at least partially on the plurality of correlation values; and classifying the heartbeat based on the maximum correlation value.

### RELATED APPLICATION

The present disclosure claims priority and other benefits from U.S. Provisional Patent Application Ser. No. 61/408,244, filed Oct. 29, 2010, entitled “MORPHOLOGY CHANGE DETECTION FOR CARDIAC SIGNAL ANALYSIS”, incorporated herein by reference in its entirety.

### CROSS-REFERENCE TO RELATED APPLICATION

Cross-reference is hereby made to the commonly-assigned related U.S. application Ser. No. ______ (attorney docket number P0036194.01), entitled “MORPHOLOGY CHANGE DETECTION FOR CARDIAC SIGNAL ANALYSIS”, to Patel et al., filed concurrently herewith and incorporated herein by reference in it's entirety.

### TECHNICAL FIELD

The disclosure relates generally to medical devices, and more particularly to analysis of cardiac signals detected by a medical device.

### BACKGROUND

An implantable medical devices (IMD) may be configured to deliver therapy and monitor physiological signals, such as cardiac signals. Some IMDs may be configured to deliver therapy in response to detection of episodes indicated by the physiological signals. The IMD may store data indicating the physiological signals, episodes identified based on the physiological signals, and therapy delivered in response to the episodes. Reviewing the data stored in the IMD memory at clinic follow-up may be desirable in order to analyze episode detection and make determinations for care of the patient. In some cases, expert knowledge is required to discriminate between correct episode detection and false episode detection. In the case of cardiac signals, for example, it may be difficult to discriminate between true ventricular arrhythmias and detection of non-ventricular arrhythmias. The effort required to review detected episodes with careful attention to detail can be burdensome.

Automatic classification of detected episodes as either true episodes or false episodes may be desirable to decrease the time required to review episodes and ensure that false detections are properly reviewed. Therefore, techniques for correctly classifying each detected episode during post-processing review of data stored in an IMD may be desirable to reduce the clinician time to review episodes, and to give the clinician increased confidence that potentially incorrect episode detections are identified and considered.

### SUMMARY

In one example, the disclosure is directed to a method and system for processing cardiac signals that includes receiving an electrogram signal that represents a heartbeat and calculating a plurality of correlation values between the heartbeat and a template heartbeat. The system and method also includes determining a maximum correlation value between the heartbeat and the template heartbeat based at least partially on the plurality of correlation values, and classifying the heartbeat based on the maximum correlation value.

In another example, the disclosure is directed to a system and method that includes receiving an electrogram signal that represents a heartbeat, and generating a heartbeat profile based on the heartbeat, wherein the heartbeat profile includes at least one measurement generated from the heartbeat. The system and method also includes comparing the heartbeat profile to a template profile generated from a template heartbeat, and determining a correlation between the heartbeat and the template heartbeat based at least partially on a comparison of the heartbeat profile and the template profile.

In another example, the disclosure is directed to a system and method that includes receiving an electrogram signal that represents a plurality of heartbeats, and identifying a group of similar heartbeats of the plurality of heartbeats. The system and method also includes generating a template heartbeat based at least partially on the group of similar heartbeats, wherein the template heartbeat is generated by calculating an average of at least some of the group of heartbeats, and comparing the template heartbeat to a heartbeat of the plurality of heartbeats that is not included in the group of similar heartbeats.

In another example, the disclosure is directed to a system and method that includes receiving an electrogram signal that represents a plurality of heartbeats, and determining a group of similar heartbeats of the plurality of heartbeats. The system and method also includes generating a first template based on a first heartbeat of the group of similar heartbeats and a second template based on a second heartbeat of the group of similar heartbeats, wherein the second heartbeat is later in time than the first heartbeat. The system and method also includes comparing the first template and the second template to a third heartbeat of the plurality of heartbeats.

In another example, the disclosure is directed to a system and method that includes receiving an electrogram signal that represents a plurality of heartbeats, and determining a first group of consecutive similar heartbeats of the plurality of heartbeats. The system and method also includes determining a second group of consecutive similar heartbeats of the plurality of heartbeats, wherein the second group of consecutive similar heartbeats is distinct from the first group of consecutive similar heartbeats. The system and method also includes identifying a transition period between the first group of consecutive similar heartbeats and the second group of consecutive similar heartbeats, wherein the transition period includes one or more heartbeats that are not similar to the heartbeats of the first group or the heartbeats of the second group.

The details of one or more examples are set forth in the accompanying drawings and the description below. Other features, objects, and advantages of the invention will be apparent from the description and drawings, and from the claims.

### BRIEF DESCRIPTION OF DRAWINGS

FIG. 1 is a conceptual diagram illustrating an example therapy system comprising an implantable medical device (IMD) for delivering stimulation therapy to a heart of a patient via implantable leads.

FIG. 2 is a block diagram illustrating an example system that includes the IMD shown in FIG. 1 connected to a programmer and access point, which is connected to a network and computing device.

FIG. 3 is a functional block diagram illustrating an example configuration of the IMD of FIG. 1.

FIG. 4 is a functional block diagram illustrating an example computing device shown in FIG. 2 having a episode classifier module.

FIG. 5 is a representation of a cardiac electrogram (EGM) having a first heartbeat pattern and a second heartbeat pattern.

FIG. 6 is a flow diagram illustrating an example method for detecting changes in EGM morphology.

FIG. 7 is a representation of an EGM having an analysis window that has been applied to a waveform for a single heartbeat having a plurality of associated profile points.

FIG. 8 is a flow diagram illustrating an example method for applying an analysis window to a heartbeat of an EGM.

FIG. 9 is a representation of analyzing a heartbeat of an EGM with a heartbeat associated with a template.

FIG. 10 is a flow diagram illustrating an example method of determining a maximum correlation between a heartbeat of an EGM and a heartbeat associated with a template.

FIG. 11 is a representation of a first heartbeat and a second heartbeat, of an EGM, that are positioned with their peaks in alignment.

FIG. 12 is a representation of a heartbeat in an EGM having an associated heartbeat profile.

FIG. 13 is a flow diagram illustrating an example method of generating a heartbeat profile and applying the heartbeat profile to a number of heartbeat templates.

FIG. 14 is a representation of an EGM having a group of similar heartbeats that are averaged together and compared to a current heartbeat under analysis.

FIG. 15 is a flow diagram illustrating an example method of comparing a heartbeat in an EGM to an average heartbeat template.

FIG. 16 is a representation of a heartbeat of an EGM being compared to a plurality of previous heartbeats.

FIG. 17 is a flow diagram illustrating an example method of comparing a heartbeat to a plurality of previous heartbeat templates.

FIG. 18 is a representation of an EGM having a transition period between groups of heartbeats.

FIG. 19 is a flow diagram illustrating an example method of analyzing heartbeats associated with a transition period.

### DETAILED DESCRIPTION

In general, aspects of the disclosure relate to an episode classifier system that can be implemented to classify cardiac episodes detected by an IMD. For example, aspects of the disclosure may relate to post-processing and automatically reviewing some or all of the cardiac episodes that were detected and stored by an IMD. Aspects of the disclosure may be implemented as part of a post processing process that retrospectively classifies episodes that were detected by an IMD. Alternatively, in some examples, such a process may be implemented in an IMD or IMD programmer. In either case, accurately classifying episodes can be useful for configuring an IMD with the appropriate parameters and therapy delivery method.

In some examples, the episode classifier techniques may include techniques for detecting changes in morphology in cardiac signals. For example, a morphology change detection algorithm can be implemented to identify changes in heartbeat patterns of an electrogram (EGM) that has been retrieved from an IMD. The morphology change detection techniques may be used to determine whether the IMD appropriately detected and classified an episode, such as a ventricular fibrillation (VF) or ventricular tachycardia (VT) episode, or inappropriately detected and classified and episode as, for example, a supraventricular tachycardia (SVT) episode. Alternatively or additionally, these techniques may be used to classify episodes in the first instance, i.e., whether an IMD previously classified the episodes or not. The morphology change detection techniques may be used to detect changes in morphology between any beats of an EGM. For example, morphology change detection techniques may be used to detect changes between consecutive beats of an EGM, beats of a first time period to a second time period, or any other beats of the EGM.

In some examples, morphology change detection techniques are used to capture individual beats of an EGM, compare the beats to other beats of the EGM or one or more known template beats to classify the beats, and group similarly classified beats together. Morphology change detection techniques are then used to identify changes in morphology using the groups of classified beats. For example, morphology change detection techniques are used to identify a change in morphology when a beat under analysis is not similar to one of the classified groups. Morphology change detection techniques may also be used to indicate, for example, whether an IMD previously classified episodes appropriately (e.g., classified a VT/VF episode as a VT/VF episode, or as an SVT episode).

According to some aspects of the disclosure, the comparison of a captured beat to a template beat can be carried out in a variety of ways. In some examples, the morphology change detection algorithm positions or “slides” a template beat over a beat under consideration, and generates a correlation value at each incremental position. The morphology change detection algorithm can then determine the position with the maximum correlation value, and use that value to classify the beat under consideration. For example, if the maximum correlation value indicates that the template beat and the beat under consideration are similar, the morphology change detection algorithm can classify the beat under consideration as being similar to the template beat. Alternatively or additionally, if the maximum correlation value indicates that the template beat and the beat under consideration are not similar (e.g., below a predetermined correlation threshold), the morphology change detection algorithm can classify the beat under consideration as being different than the template beat.

In other examples, the morphology change detection algorithm may compare a beat under consideration to a template beat by positioning the template beat in a plurality of specific positions with respect to the beat under consideration, and generate a correlation value at each of the designated positions. For example, the system may align the beats in three positions: (1) with the Q points of the beats aligned (2) with the R points of the beats aligned, and (3) with the S points of the beats aligned. The morphology change detection algorithm may then select the position with the maximum correlation value, and use that value to classify the beat under consideration. For example, the morphology change detection algorithm can use the maximum correlation value to classify the beat under consideration as similar or dissimilar to the template beat.

In another aspect of the present disclosure, the morphology change detection algorithm may compare a beat under consideration to a template beat by generating a profile of a captured beat, comparing the profile of the captured beat to a template beat profile, and classifying the captured beat according to the comparison of the profiles. For example, the morphology change detection algorithm may generate a profile of a beat based on distances between inflection points of the beat (e.g., horizontal and/or vertical distances), slope values associated with the beat, frequency content of the beat or other characteristics such as the amplitudes of the P, Q, R, and S points of the beat, or width of the beat. The morphology change detection algorithm can then compare the profile of a beat under consideration to a template beat profile, and use that comparison to classify the beat under consideration.

In some examples, the morphology change detection algorithm can use the profile comparison and the maximum template correlation value independently or in tandem. For example, according to some aspects of the disclosure, the morphology change detection algorithm can compare or combine a maximum correlation value and a profile correlation value or score to classify a beat under consideration.

In another aspect of the present disclosure, after the morphology change detection algorithm classifies the beats according to one or more templates, the morphology change detection algorithm groups the beats according to their classifications. Grouping beats may help to prevent false identifications of morphology changes. For example, beats of an EGM may change slightly over time. Slight changes in beat shape may be exaggerated if the morphology change detection algorithm compares two beats that are not close in time (e.g., a template beat generated from a first beat, and a second beat (under consideration) later in time). As such, if the morphology change detection algorithm generates a template beat based on the first beat of an EGM and applies that template to all of the beats of the EGM, a beat later in time may not closely correlate with the template beat, even though the beat is the same type of beat as the template beat. Grouping beats and generating templates according to the groups can smooth inconsistencies in beats over time.

In some examples, the morphology change detection algorithm averages all beats of a group of beats to produce a representative template beat for the group. For example, all beats classified as belonging to a certain template are averaged (e.g., the peaks of the beats are averaged) to produce a single template beat that inherently includes characteristics of all the contributing beats. The morphology change detection algorithm can then compare the average template beat to a beat under analysis, such as for example, by determining a maximum correlation value or comparing a profile of the average template beat to a profile of the beat under analysis.

In some other examples, the morphology change detection algorithm may select a plurality of beats from a group of beats to compare to a beat under analysis. For example, morphology change detection algorithm may select the first beat of a group and the last beat added to the group for comparison to a beat under analysis. The morphology change detection algorithm can then update (e.g., select new) templates for each beat being analyzed. For example, when a beat is classified as being part of a group, the morphology change detection algorithm compares the newly added beat to the next beat under analysis.

In another aspect of the disclosure, the morphology change detection algorithm identifies a transition period between a first group of beats and a second group of beats. Identifying a transition period may aid in determining where a first predominant morphology ends and a second prominent morphology begins. For example, the transition period provides for a period of ectopy, so that the morphology change detection algorithm does not identify several changes in morphology in short succession when there is truly only a single change in morphology.

In some examples, the morphology change detection algorithm may remove certain beats of the transition period from consideration to avoid a false detection of multiple morphology changes. For example, the morphology change detection algorithm determines the transition period by identifying a leftmost beat of beats belonging to a first morphology and a rightmost beat of beats belonging to a second morphology. The morphology change detection algorithm then sets the transition period to include all beats between the identified leftmost and rightmost beats, as these are the beats that the system identifies as being irregular in nature.

After identifying the transition period, the morphology change detection algorithm may then remove beats from consideration. For example, the morphology change detection algorithm determines which beat within the transition period has the fewest consecutive beats, and removes that beat from consideration. If no beat has more consecutive beats than another beat, all beats are removed consideration. The morphology change detection algorithm repeats the removal process until all beats of the transition period are similar. By recursively removing beats in the transition period, the morphology change detection algorithm can identify the boundaries of a transition period where the beats transition from one morphology to another.

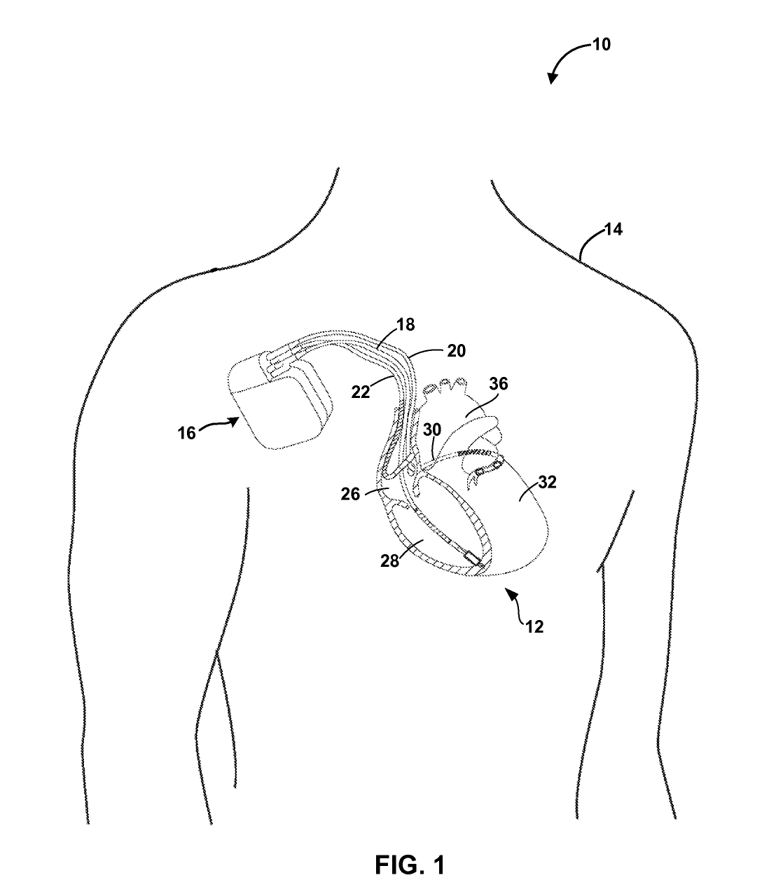

FIG. 1 is a conceptual diagram illustrating an example therapy system 10 that may be used to provide therapy to heart 12 of patient 14. Therapy system 10 includes IMD 16, which is coupled to leads 18, 20, and 22. IMD 16 may be, for example, an implantable pacemaker, cardioverter, and/or defibrillator that provides electrical signals to heart 12 via electrodes coupled to one or more of leads 18, 20, and 22. Patient 12 is ordinarily, but not necessarily, a human patient.

Leads 18, 20, 22 extend into the heart 12 of patient 14 to sense electrical activity of heart 12 and/or deliver electrical stimulation to heart 12. In the example shown in FIG. 1, right ventricular (RV) lead 18 extends through one or more veins (not shown), the superior vena cava (not shown), and right atrium 26, and into right ventricle 28. Left ventricular (LV) coronary sinus lead 20 extends through one or more veins, the vena cava, right atrium 26, and into the coronary sinus 30 to a region adjacent to the free wall of left ventricle 32 of heart 12. Right atrial (RA) lead 22 extends through one or more veins and the vena cava, and into right atrium 26 of heart 12. In some alternative embodiments, therapy system 10 may include an additional lead or lead segment (not shown in FIG. 1) that deploys one or more electrodes within the vena cava or other vein. These electrodes may allow alternative electrical sensing configurations that may provide improved sensing accuracy in some patients.

IMD 16 may sense electrical signals attendant to the depolarization and repolarization of heart 12 via electrodes (not shown in FIG. 1) coupled to at least one of the leads 18, 20, 22. In some examples, IMD 16 provides pacing pulses to heart 12 based on the electrical signals sensed within heart 12. The configurations of electrodes used by IMD 16 for sensing and pacing may be unipolar or bipolar. IMD 16 may also provide defibrillation therapy and/or cardioversion therapy via electrodes located on at least one of the leads 18, 20, 22. IMD 16 may detect arrhythmia of heart 12, such as fibrillation of ventricles 28 and 32, and deliver cardioversion or defibrillation therapy to heart 12 in the form of electrical shocks. In some examples, IMD 16 may be programmed to deliver a progression of therapies, e.g., pulses with increasing energy levels, until a tachyarrhythmia of heart 12 is stopped. IMD 16 may detect tachycardia or fibrillation employing one or more tachycardia or fibrillation detection techniques known in the art.

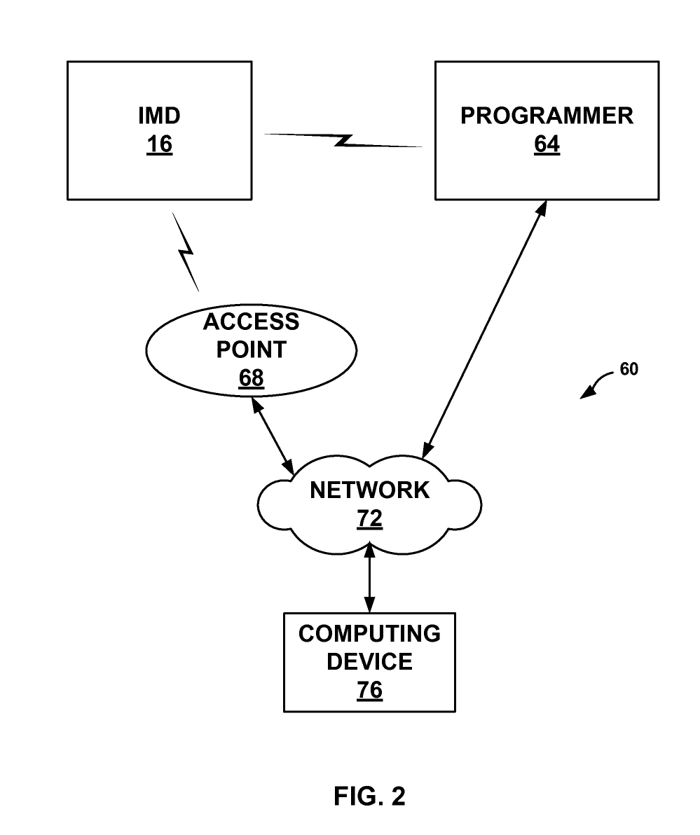

FIG. 2 is a block diagram illustrating an example system 60 that includes IMD 16 shown in FIG. 1, as well as programmer 64, access point 68, a network 72, and a computing device 76. As shown in FIG. 2, the IMD 16 is connected to programmer 24 and access point 68. Access point 68 connects the IMD 16 to computing device 76 via network 72.

In some examples, programmer 64 may be a handheld computing device, computer workstation, or networked computing device. Programmer 64 may include a user interface that receives input from a user. The user interface may include, for example, a keypad and a display, which may for example, be a cathode ray tube (CRT) display, a liquid crystal display (LCD) or light emitting diode (LED) display. The keypad may take the form of an alphanumeric keypad or a reduced set of keys associated with particular functions. Programmer 64 can additionally or alternatively include a peripheral pointing device, such as a mouse, via which a user may interact with the user interface. In some embodiments, a display of programmer 64 may include a touch screen display, and a user may interact with programmer 64 via the display. It should be noted that the user may also interact with programmer 64 or IMD 16 remotely via networked computing device 76.

A user, such as a physician, technician, surgeon, electrophysiologist, or other clinician, may interact with programmer 64 to communicate with IMD 16. For example, the user may interact with programmer 64 to retrieve physiological or diagnostic information from IMD 16. A user may also interact with programmer 64 to program IMD 16, e.g., select values for operational parameters of IMD 16.

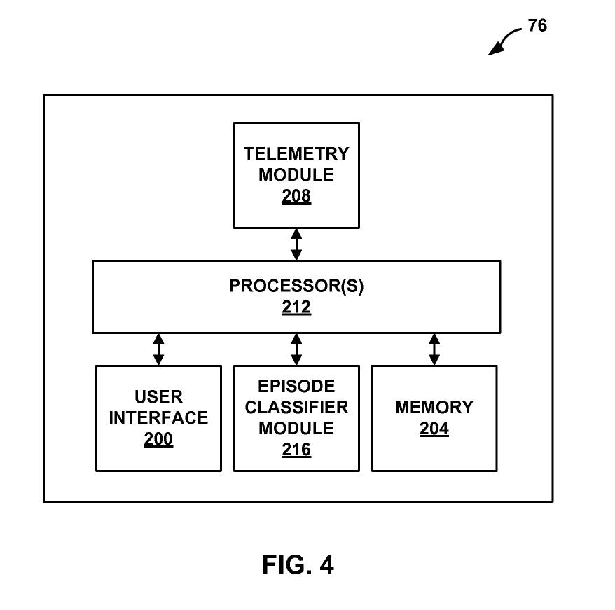

For example, the user may use programmer 64 to retrieve information from IMD 16 regarding the rhythm of heart 12, trends therein over time, or arrhythmic episodes. The information may include EGM data, marker channel data, or the like. In some examples, the information retrieved from IMD 16 may be further processed by an episode classifier module, as shown in FIG. 4. As another example, the user may use programmer 64 to retrieve information from IMD 16 regarding other sensed physiological parameters of patient 14, such as intracardiac or intravascular pressure, activity, posture, respiration, or thoracic impedance. As another example, the user may use programmer 64 to retrieve information from IMD 16 regarding the performance or integrity of IMD 16 or other components of system 10, such as leads 18, 20 and 22, or a power source of IMD 16.

The user may use programmer 64 to program a therapy progression, select electrodes used to deliver defibrillation pulses, select waveforms for the defibrillation pulses, or select or configure a fibrillation detection algorithm for IMD 16. The user may also use programmer 64 to program similar aspects of other therapies provided by IMD 16, such as cardioversion or pacing therapies. In some examples, the user may activate certain features of IMD 16 by entering a single command via programmer 64, such as depression of a single key or combination of keys of a keypad or a single point-and-select action with a pointing device.

IMD 16 and programmer 64 may communicate via wireless communication using any techniques known in the art. Examples of communication techniques may include, for example, low frequency inductive telemetry or radiofrequency (RF) telemetry, but other techniques are also contemplated. In some examples, programmer 64 may include a programming head that may be placed proximate to the patient's body near the IMD 16 implant site in order to improve the quality or security of communication between IMD 16 and programmer 64.

IMD 16 is an example of a device that may store electrograms (EGMs) that are associated with sensed episodes or events that may be non-physiological and, instead, associated with a sensing integrity condition. Such EGMs may be retrieved from IMD 16 by programmer 64, and displayed by programmer 64 for evaluation by a clinician or other user to, for example, determine whether an episode sensed by the IMD 16 has been appropriately classified by the IMD 16. For example, the clinician or other user can determine whether episodes classified by IMD 16 as VT/VF episodes were classified appropriately. The EGMs may be considered in conjunction within other sensing integrity data, such as lead impedance data, which may also be stored by IMD 16, and retrieved and displayed by programmer 64. The EGMs may be stored with respective marker channels.

In other examples, one or more devices other than IMD 16 may, alone, or in combination with IMD, implement the techniques described herein. For example, programmer 64 or another external device may store EGMs based on cardiac signal data received from IMD 16. Programmer 64 or another external device may determine whether to store the EGMs, according to any of the techniques described herein, based on the cardiac signal or other signals or information received from IMD 16. Furthermore, in some examples, the medical device and/or leads are not implanted.

As described above, IMD 16 is also connected to access point 68 and computing device 76 via network 72. In the example of FIG. 2, access point 68, programmer 64, and computing device 76 are interconnected, and able to communicate with each other, through network 72. Access point 68 may comprise a device that connects to network 72 via any of a variety of connections, such as telephone dial-up, digital subscriber line (DSL), or cable modem connections. In other embodiments, access point 68 may be coupled to network 72 through different forms of connections, including wired or wireless connections. In some examples, access point 68 may be co-located with patient 14 and may comprise one or more programming units and/or computing devices (e.g., one or more monitoring units) that may perform various functions and operations described herein. For example, access point 68 may include a home-monitoring unit that is co-located with patient 14 and that may monitor the activity of IMD 16.

Network 72 may comprise a local area network, wide area network, or global network, such as the Internet. In some cases, programmer 64 and/or access point 68 may assemble episode logs, including EGMs, and other sensing integrity information in web pages or other documents for viewing by trained professionals, such as clinicians. The trained professionals may analyze the information via viewing terminals associated with a computing device, such as computing device 76. In some examples, computing device 76 may be equipped to execute a post processing program that identifies and classifies episodes contained in the episode logs. System 60 may be implemented, in some aspects, with general network technology and functionality similar to that provided by the Medtronic CareLink® Network developed by Medtronic, Inc., of Minneapolis, Minn.

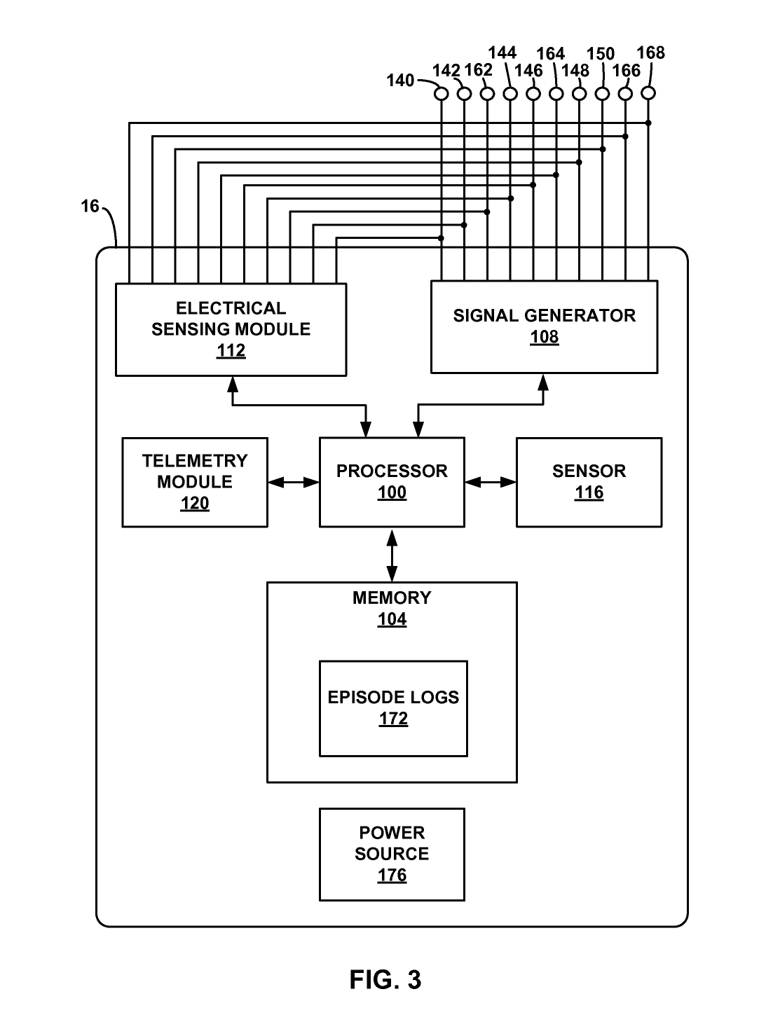

FIG. 3 is a functional block diagram illustrating one example configuration of IMD 16. In the example illustrated by FIG. 4, IMD 16 includes a processor 100, memory 104, signal generator 108, electrical sensing module 112, sensor 116, telemetry module 120, and power source 124. Memory 104 may includes computer-readable instructions that, when executed by processor 100, cause IMD 16 and processor 100 to perform various functions attributed to IMD 16 and processor 100 herein. Memory 104 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Processor 100 may include any one or more of a microprocessor, a controller, a digital signal processor (DSP), an application specific integrated circuit (ASIC), a field-programmable gate array (FPGA), or equivalent discrete or integrated logic circuitry. In some examples, processor 100 may include multiple components, such as any combination of one or more microprocessors, one or more controllers, one or more DSPs, one or more ASICs, or one or more FPGAs, as well as other discrete or integrated logic circuitry. The functions attributed to processor 100 herein may be embodied as software, firmware, hardware or any combination thereof.

Processor 100 controls signal generator 108 to deliver stimulation therapy to heart 12. Processor 100 may control signal generator 108 to deliver stimulation according to a selected one or more therapy programs, which may be stored in memory 104. For example, processor 100 may control signal generator 108 to deliver electrical pulses with the amplitudes, pulse widths, frequency, or electrode polarities specified by the selected one or more therapy programs.

Signal generator 108 is electrically coupled to electrodes 140, 142, 144, 146, 148, 150, 158, 162, 164, and 166, e.g., via conductors of the respective lead 18, 20, 22 of IMD 16. Signal generator 108 configured to generate and deliver electrical stimulation therapy to heart 12. For example, signal generator 108 may deliver defibrillation shocks to heart 12 via at least two electrodes 158, 162, 164, 166. Signal generator 108 may deliver pacing pulses via ring electrodes 140, 144, 148 coupled to leads 18, 20, and 22, respectively, and/or helical electrodes 142, 146, and 150 of leads 18, 20, and 22, respectively. In some examples, signal generator 108 delivers pacing, cardioversion, or defibrillation stimulation in the form of electrical pulses or shocks. In other examples, signal generator 108 may deliver one or more of these types of stimulation in the form of other signals, such as sine waves, square waves, or other substantially continuous time signals.

Signal generator 108 may include a switch module and processor 100 may use the switch module to select, e.g., via a data/address bus, which of the available electrodes are used to deliver pacing, cardioversion, or defibrillation pulses or shocks. The switch module may include a switch array, switch matrix, multiplexer, or any other type of switching device suitable to selectively couple stimulation energy to selected electrodes.

Electrical sensing module 112 monitors signals from at least one of electrodes 140, 142, 144, 146, 148, 150, 158, 162, 164 or 166 in order to monitor electrical activity of heart 12. Electrical sensing module 86 may also include a switch module to select which of the available electrodes are used to sense the heart activity. In some examples, processor 80 may select the electrodes that function as sense electrodes, or the sensing electrode configuration, via the switch module within electrical sensing module 86, e.g., by providing signals via a data/address bus. Electrical sensing module 86 may include multiple detection channels, each of which may comprise a sense amplifier. In response to the signals from processor 80, the switch module of within electrical sensing module 86 may couple selected electrodes to each of the detection channels.

If IMD 16 is configured to generate and deliver pacing pulses to heart 12, processor 100 may include pacer timing and control module, which may be embodied as hardware, firmware, software, or any combination thereof. The pacer timing and control module may comprise a dedicated hardware circuit, such as an ASIC, separate from other components of processor 100, such as a microprocessor, or a software module executed by a component of processor 100, which may be a microprocessor or ASIC. The pacer timing and control module may include programmable counters which control the basic time intervals associated with DDD, VVI, DVI, VDD, AAI, DDI, DDDR, VVIR, DVIR, VDDR, AAIR, DDIR and other modes of single and dual chamber pacing. In the aforementioned pacing modes, “D” may indicate dual chamber, “V” may indicate a ventricle, “I” may indicate inhibited pacing (e.g., no pacing), and “A” may indicate an atrium. The first letter in the pacing mode may indicate the chamber that is paced, the second letter may indicate the chamber that is sensed, and the third letter may indicate the chamber in which the response to sensing is provided.

Intervals defined by the pacer timing and control module within processor 100 may include atrial and ventricular pacing escape intervals, refractory periods during which sensed P-waves and R-waves are ineffective to restart timing of the escape intervals, and the pulse widths of the pacing pulses. As another example, the pace timing and control module may define a blanking period, and provide signals to electrical sensing module 112 to blank one or more channels, e.g., amplifiers, for a period during and after delivery of electrical stimulation to heart 12. The durations of these intervals may be determined by processor 100 in response to stored data in memory 104. The pacer timing and control module of processor 100 may also determine the amplitude of the cardiac pacing pulses.

During pacing, escape interval counters within the pacer timing/control module of processor 100 may be reset upon sensing of R-waves and P-waves with detection channels of electrical sensing module 112. Signal generator 108 may include pacer output circuits that are coupled, e.g., selectively by a switching module, to any combination of electrodes 140, 142, 144, 146, 148, 150, 158, 162, or 166 appropriate for delivery of a bipolar or unipolar pacing pulse to one of the chambers of heart 12. Processor 100 may reset the escape interval counters upon the generation of pacing pulses by signal generator 108, and thereby control the basic timing of cardiac pacing functions, including anti-tachyarrhythmia pacing.

The value of the count present in the escape interval counters when reset by sensed R-waves and P-waves may be used by processor 100 to measure the durations of R-R intervals, P-P intervals, P-R intervals and R-P intervals, which are measurements that may be stored in memory 104. Processor 100 may use the count in the interval counters to detect a tachyarrhythmia event, such as an atrial or ventricular fibrillation (VF) or ventricular tachycardia (VT).

In some examples, processor 100 may operate as an interruptdriven device that is responsive to interrupts from pacer timing and control module, where the interrupts may correspond to the occurrences of sensed P-waves and R-waves and the generation of cardiac pacing pulses. Any necessary mathematical calculations to be performed by processor 100 and any updating of the values or intervals controlled by the pacer timing and control module of processor 100 may take place following such interrupts. A portion of memory 104 may be configured as a plurality of recirculating buffers, capable of holding series of measured intervals, which may be analyzed by processor 100 in response to the occurrence of a pace or sense interrupt to determine whether the patient's heart 12 is presently exhibiting atrial or ventricular tachyarrhythmia.

In some examples, an arrhythmia detection method may include any suitable tachyarrhythmia detection algorithms. In one example, processor 100 may utilize all or a subset of the rule-based detection methods described in U.S. Pat. No. 5,545,186 to Olson et al., entitled, “PRIORITIZED RULE BASED METHOD AND APPARATUS FOR DIAGNOSIS AND TREATMENT OF ARRHYTHMIAS,” which issued on Aug. 13, 1996, in U.S. Pat. No. 5,755,736 to Gillberg et al., entitled, “PRIORITIZED RULE BASED METHOD AND APPARATUS FOR DIAGNOSIS AND TREATMENT OF ARRHYTHMIAS,” which issued on May 26, 1998, or in U.S. patent application Ser. No. 10/755,185, filed Jan. 8, 2004 by Kevin T. Ousdigian, entitled “REDUCING INAPPROPRIATE DELIVERY OF THERAPY FOR SUSPECTED NON-LETHAL ARRHYTHMIAS.” U.S. Pat. No. 5,545,186 to Olson et al., U.S. Pat. No. 5,755,736 to Gillberg et al., and U.S. patent application Ser. No. 10/755,185 by Kevin T. Ousdigian are incorporated herein by reference in their entireties. However, other arrhythmia detection methodologies may also be employed by processor 100 in other examples.

In the event that processor 100 detects an atrial or ventricular tachyarrhythmia based on signals from electrical sensing module 112, and an anti-tachyarrhythmia pacing regimen is desired, timing intervals for controlling the generation of anti-tachyarrhythmia pacing therapies by signal generator 108 may be loaded by processor 100 into the pacer timing and control module to control the operation of the escape interval counters therein and to define refractory periods during which detection of R-waves and P-waves is ineffective to restart the escape interval counters.

If IMD 16 is configured to generate and deliver defibrillation shocks to heart 12, signal generator 108 may include a high voltage charge circuit and a high voltage output circuit. In the event that generation of a cardioversion or defibrillation shock is required, processor 100 may employ the escape interval counter to control timing of such cardioversion and defibrillation shocks, as well as associated refractory periods. In response to the detection of atrial or ventricular fibrillation or tachyarrhythmia requiring a cardioversion shock, processor 100 may activate a cardioversion/defibrillation control module, which may, like the pacer timing and control module, be a hardware component of processor 100 and/or a firmware or software module executed by one or more hardware components of processor 100. The cardioversion/defibrillation control module may initiate charging of the high voltage capacitors of the high voltage charge circuit of signal generator 108 under control of a high voltage charging control line.

Processor 100 may monitor the voltage on the high voltage capacitor, e.g., via a voltage charging and potential (VCAP) line. In response to the voltage on the high voltage capacitor reaching a predetermined value set by processor 100, processor 100 may generate a logic signal that terminates charging. Thereafter, timing of the delivery of the defibrillation or cardioversion pulse by signal generator 108 is controlled by the cardioversion/defibrillation control module of processor 100. Following delivery of the fibrillation or tachycardia therapy, processor 100 may return signal generator 108 to a cardiac pacing function and await the next successive interrupt due to pacing or the occurrence of a sensed atrial or ventricular depolarization.

Signal generator 108 may deliver cardioversion or defibrillation shocks with the aid of an output circuit that determines whether a monophasic or biphasic pulse is delivered, whether housing electrode 158 serves as cathode or anode, and which electrodes are involved in delivery of the cardioversion or defibrillation pulses. Such functionality may be provided by one or more switches or a switching module of signal generator 108.

IMD 16 may comprise one or more sensors, such as sensor 116 illustrated in the example of FIG. 3. Sensor 116 may be located on or within on or more of leads 18, 20 and 22, or another lead which may or may not include stimulation/sensing electrodes. In some examples, sensor 116 may be separately housed from IMD 16, and may be coupled to IMD 16 via wireless communication. Sensor 116 may be implanted or external.

Sensor 116 may comprise, as examples, a pressure sensor, a motion sensor, a heart sound sensor, or any sensor capable of generating a signal that varies a function of mechanical activity, e.g., contraction, of heart 12. A pressure sensor may be, for example, a capacitive pressure sensor that senses an intracardiac or other cardiovascular pressure. A motion sensor may be, for example, an accelerometer or piezoelectric element. Processor 100 may receive one or more signals from sensor 116 or a plurality of sensors. Processor 100 may monitor, among other things, the mechanical activity of heart 12 based on such signals.

Telemetry module 120 includes any suitable hardware, firmware, software or any combination thereof for communicating with another device, such as programmer 64 (FIG. 2). Under the control of processor 100, telemetry module 120 may receive downlink telemetry from and send uplink telemetry to programmer 64 with the aid of an antenna, which may be internal and/or external. Processor 100 may provide the data to be uplinked to programmer 64 and the control signals for the telemetry circuit within telemetry module 120, e.g., via an address/data bus.

In some examples, processor 100 may transmit atrial and ventricular heart signals (e.g., EGM signals) produced by atrial and ventricular sense amp circuits within electrical sensing module 112 to programmer 64 and/or computing device 76. Programmer 64 and/or computing device 76 may interrogate IMD 16 to receive the EGMs and/or other data. Processor 100 may store EGMs within memory 104, and retrieve stored EGMs from memory 104. Processor 100 may also generate and store marker channel codes indicative of different cardiac events that electrical sensing module 112 detects, such as ventricular and atrial depolarizations, and transmit the marker codes to programmer 64. In some examples, the marker codes are further processed by a post processing device (e.g., the programmer 64 or computing device 76) having an episode classifier. The post processing device may be used, for example, to verify cardiac events sensed by the electrical sensing module 112. An example pacemaker with marker-channel capability is described in U.S. Pat. No. 4,374,382 to Markowitz, entitled, “MARKER CHANNEL TELEMETRY SYSTEM FOR A MEDICAL DEVICE,” which issued on Feb. 15, 1983 and is incorporated herein by reference in its entirety.

In some examples, processor 100 may perform a morphological analysis on the EGM to characterize the beats of the EGM. For example, a morphological analysis may include any one or more of an amplitude regularity analysis, an analysis of the width of the QRS complex or other features of the EGM, or an analysis of slew rates. In some examples, a morphological analysis may involve a wavelet analysis, such as those described in U.S. Pat. No. 6,393,316, entitled “METHOD AND APPARATUS FOR DETECTION AND TREATMENT OF CARDIAC ARRHYTHMIAS,” which issued to Gillberg et al. on May 21. 2002, and U.S. Pat. No. 7,176,747, entitled “IDENTIFICATION OF OVERSENSING USING SINUS R-WAVE TEMPLATE,” which issued to Gunderson et al. on Jan. 23, 2007. In some examples, the analysis may include the far-field EGM analysis techniques described in U.S. Pat. No. 7,333,855 to Gunderson et al., entitled “METHOD AND APPARATUS FOR DETERMINING OVERSENSING IN A MEDICAL DEVICE,” which issued on Feb. 19, 2008. The entire content of each of U.S. Pat. Nos. 6,393,316, 7,176,747 and 7,333,855 is incorporated herein by reference in its entirety.

Processor 100 may store cardiac EGMs for physiological episodes, such as tachyarrhythmias, within episode logs 172 in memory 104. For example, processor 100 may store cardiac EGMs for atrial and ventricular tachycardia (VT) and ventricular fibrillation (VF) episodes, in response to the detection of the tachycardia or fibrillation using any of the techniques described above. The EGM may include data collected by the IMD during detection of the tachyarrhythmia, as well as after detection, e.g., during treatment of the tachyarrhythmia. The data stored for the episode may also include a marker channel associated with the EGM. The marker channel may annotate the EGM with events detected by the IMD, such as ventricular or atrial depolarizations, as well an indication of when during the episode a responsive therapy was delivered by the IMD.

The various components of IMD 16 are coupled to power source 176, which may include a rechargeable or non-rechargeable battery. A non-rechargeable battery may be capable of holding a charge for several years, while a rechargeable battery may be inductively charged from an external device, e.g., on a daily or weekly basis.

FIG. 4 is a block diagram of a computing device, such as computing device 76 shown in FIG. 2. It should be noted that certain functions and computations described as being carried out by computing device 76 may also be carried out by, independently or in conjunction with, programmer 64. As shown in FIG. 4, computing device 76 includes user interface 200, memory 204, telemetry module 208, one or more processors 212, and episode classifier module 216.

User interface 200 allows a user to interact with computing device 76. Examples of user interface 200 include a keypad embedded on computing device 76, a keyboard, a mouse, a roller ball, buttons, or other devices that allow a user to interact with computing device 76. Memory 204 stores instructions for applications that may be executed by one or more processors 212. One or more processors 212 may include any one or more of a microprocessor, a controller, a digital signal processor (DSP), an application specific integrated circuit (ASIC), a field-programmable gate array (FPGA), or equivalent discrete or integrated logic circuitry. Additionally, the functions attributed to processor 212, in this disclosure, may be embodied as software, firmware, hardware or any combination thereof. For purposes of illustration only, in the following description, applications that may be executed by one or more processors 212 are described below as being executed by processor 212. The applications may be executed by processor 212 in response to a user interacting with user interface 200 to execute the applications. For example, processor 212 may execute a post processing application for analyzing EGMs that have been downloaded from an IMD, such as IMD 16 in response to a user launching the post processing application.

Memory 204 may also include instructions that cause processor 212 to perform various functions ascribed to processor 212 in this disclosure. Memory 204 may comprise a computer-readable, machine-readable, or processor-readable storage medium that comprises instructions that cause one or more processors, e.g., processor 212, to perform various functions. Memory 204 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. In some embodiments, memory 204 may comprise one or more of a non-transitory/tangible storage media, where the data stored in such media may or may not change (e.g., ROM, RAM).

Telemetry module 208 includes any suitable hardware, firmware, software or any combination thereof for communicating with another device, such as IMD 16 (FIGS. 1 and 2). Under the control of processor 212, telemetry module 208 may receive downlink telemetry from and send uplink telemetry to IMD 16 with the aid of an antenna, which may be internal and/or external. Processor 212 may provide the data received from IMD 16 to memory 204 or episode classifier module 216 via an address/data bus.

In some examples, episode classifier module 216 may apply one or more algorithms and/or execute other instructions for processing EGMs, electrocardiograms (ECGs) or other signals generated by a heart monitoring or pacing apparatus. As described above, IMD 16 may perform a variety of analyses to identify episodes of arrhythmia and/or tachyarrhythmia. In particular, IMD 16 may identify episodes of ventricular tachycardia and/or ventricular fibrillation and deliver appropriate therapy, such as pacing, cardioversion or defibrillation therapy. However, it may be desirable to verify that episodes detected and identified by IMD 16 were properly identified and characterized. For example, in some instances, the analyses executed by processor 100 of IMD 16 may improperly identify an episode due to sensing, processing, time or other limitations. In such cases, a trained professional, such as a clinician or doctor may wish to analyze EGMs from IMD 16 further using episode classifier module 216 of computing device 76. The data gathered during this “post processing” (e.g., upon retrieving and processing episode data including EGM data, marker channel data, episode detection data, or other data from IMD 16) can be used, for example, by the trained professional to adjust the manner in which IMD 16 provides therapy.

Episode classifier module 216 may, in some examples, be used for post-processing to identify changes in morphology and to indicate a particular type of episode. For example, changes in morphologies at the onset of an arrhythmia may indicate ventricular tachycardia (VT) and/or ventricular fibrillation (VF). In some examples, episode classifier module 216 can include a morphology change detection algorithm that retrospectively determines if a ventricular morphology change occurred at the onset of an arrhythmia. As described in greater detail below, the morphology change detection algorithm may utilize EGMs and marker data to identify morphology changes. While the episode classifier module 216 is described herein as processing EGMs during a post-processing phase, in other examples, the techniques ascribed to classifier module 216 may be carried out an IMD (e.g., such as IMD 16), for example, to delivery an appropriate therapy.

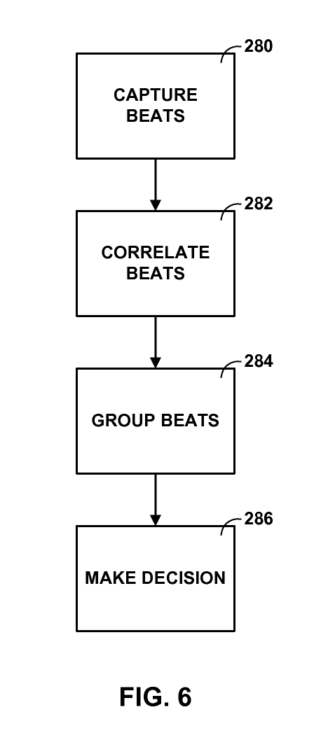

According to some aspects of the disclosure, episode classifier module 216 may utilize techniques for analyzing EGMs described with respect to FIGS. 6-19 below for identifying changes in morphology. For example, according to an aspect of the disclosure, episode classifier module 216 captures individual beats of an EGM, compares the captured beats to a template beat, and classifies the beat according to the comparison. In some examples, episode classifier module 216 compares captured beats to template beats by positioning or “sliding” the template beat over a beat under consideration, and generating a correlation value at each incremental position. In other examples, episode classifier module 216 positions a template beat in a plurality of specific positions with respect to a beat under consideration, and generates a correlation value at each of the designated positions. For example, episode classifier module 216 may align the beats according to the P, Q, R, and/or S points of the beats, according to the maximum or minimum values of the beats, or according to other beat characteristics.

According to another aspect of the disclosure, episode classifier module 216 captures individual beats of an EGM, generates a profile of a captured beat, compares the profile of the captured beat to a template beat profile, and classifies the captured beat according to the comparison of the profiles. For example, episode classifier module 216 generates a profile of a beat based on distances between inflection points of the beat (e.g., horizontal and/or vertical distances), or other characteristics such as the amplitudes of the P, Q, R, and S points of the beat. Inflection points can be identified, for example, by monitoring the EGM signal and determining when the signal transitions between an increasing signal (e.g., increasing voltage) and a decreasing signal (e.g., decreasing voltage). Episode classifier module 216 then compares the profile of a beat under consideration to a template beat profile, and uses that comparison to classify the beat under consideration.

According to another aspect of the invention, after comparing and classifying beats, episode classifier module 216 groups the beats according to their classifications. In some examples, episode classifier module 216 averages all beats of a group of beats to produce a representative template beat for the group. For example, all beats classified as belonging to a certain template are averaged (e.g., the peaks of the beats are averaged) to produce a single template beat that inherently includes characteristics of all the contributing beats. In some other examples, episode classifier module 216 dynamically selects a plurality of beats from a group of beats to compare to a beat under analysis. For example, morphology change detection algorithm may select the first beat of a group and the last beat added to the group for comparison to a beat under analysis.

According to another aspect of the disclosure, episode classifier module 216 identifies a transition period between a first group of beats and a second group of beats. For example, episode classifier module 216 recursively removes certain beats of the transition period from consideration to avoid a false detection of multiple morphology changes. By recursively removing beats in the transition period, episode classifier module 216 can identify boundaries of a transition period where the beats transition from one morphology to another.

While certain techniques are described herein as being carried out by episode classifier module 216, in other examples, such methods and processes may be carried out by processor 212. For example, certain techniques ascribed as being carried out by episode classifier module 216 may be carried out by processor 212. In addition, while episode classifier module 216 is described as being included in computing device 76, in other examples, episode classifier module 216 may be incorporated in programmer 64 or IMD 16.

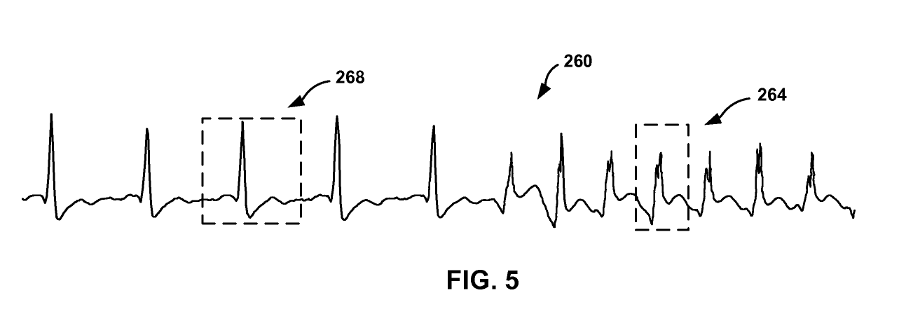

FIG. 5 is a representation of an EGM 260 having a first heartbeat pattern 264 and a second heartbeat pattern 268. EGM 260 may be generated, for example, by IMD 16 (FIGS. 2 and 3) and stored in episode logs 172. In addition, EGM 260 may be transferred from episode logs 172 to computing device 76 or programmer 64 for post processing. For example, in some examples, EGM 260 can be analyzed using episode classifier module 216 (FIG. 4) to identify morphology changes. As described above, changes in heartbeats can be used to signal an arrhythmia and, more particularly, a VT episode or VF episode. In particular, analysis of the EGM waveform may permit discrimination between VT/VF episodes and supraventricular tachycardia (SVT) episodes. In some examples, the EGM 260 may be analyzed by episode classifier module 216 of computing device 76.

FIG. 6 is a flow diagram illustrating an example method for detecting changes in morphology. In some examples, the method shown in FIG. 6 may be carried out by computing device 76. For example, the method shown in FIG. 6 may be carried out by episode classifier module 216 to detect changes in morphology. Accordingly, for purposes of illustration only, the method of FIG. 6 is described with respect to computing device 76 shown in FIG. 4, though various other systems and/or devices may be utilized to implement or perform the method shown in FIG. 6. For example, in some other examples, the method shown in FIG. 6 may be carried out by programmer 64 (FIG. 2) or IMD 16 (FIG. 1).

In some aspects of the disclosure, the method of FIG. 6 is used for processing an EGM. For example, an IMD, such as IMD 16, can be used to monitor the heartbeat of a patient and generate electrical signal(s) based on the heartbeat. IMD 16 can convert the signals to produce a digital waveform having a plurality of samples (e.g., analog to digital converting of the electrical signals). According to the method shown in FIG. 6, computing device 76 captures individual beats (e.g., a number of digital samples representing a single beat) of an EGM for further analysis (280). Capturing individual beats and separating them from the other beats of the EGM allows the beats to be compared to one or more known, template beats (e.g., digital samples of a known heartbeat waveform). Computing device 76 may capture the beats, for example, by setting a “window” around the individual beats, thereby separating one beat from the other beats of the EGM. In some examples, computing device 76 sets the window according to a predetermined number of EGM samples around a particular characteristic of a beat. For example, a beat include a marker or other identifying characteristic (e.g., a peak of a beat). Computing device 76 can set a symmetric window, then, by counting a certain number of EGM samples on both sides of the identifying characteristic (e.g., 10 samples on each side of the identifying characteristic, 15 samples on each side of the identifying characteristic, 30 samples on each side of the identifying characteristic, etc). In other examples, computing device 76 can also set an asymmetric window around an identifying characteristic of a beat. For example, computing device 76 may set a window that is 10 samples prior to an R-wave point of the beat, and 20 samples after the R-wave point of the beat. By setting an asymmetrical window around a certain identifying characteristic of the beat (e.g., the R-wave), the computing device 76 can focus on morphological changes associated with a certain portion of the beat.

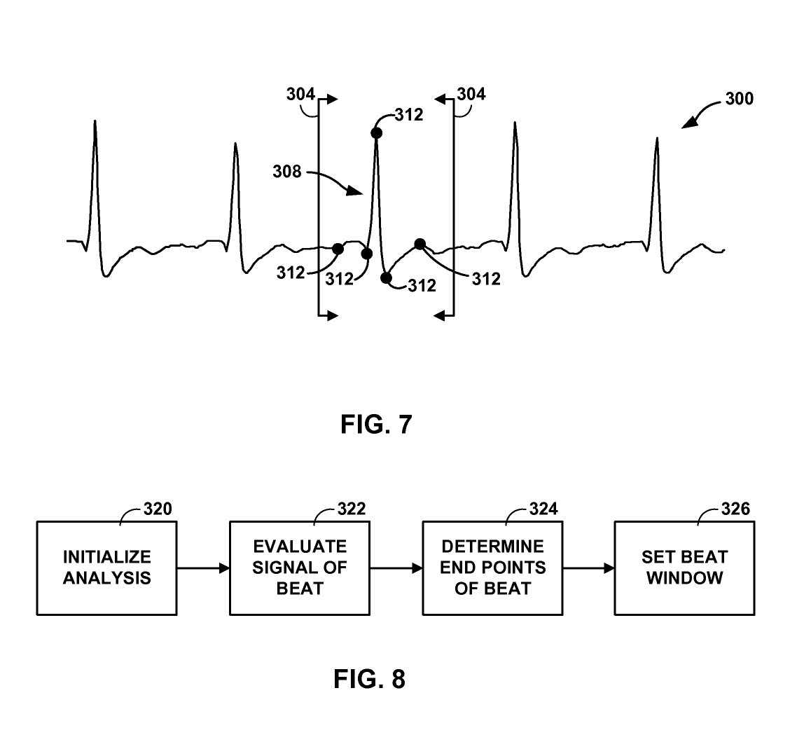

In other examples, as described in greater detail with respect to FIGS. 7 and 8, the window around a particular beat may be determined according to characteristics of the beat. For example, computing device 76 may have the ability to apply a customizable window around a beat being captured, according to the characteristics the beat. In some examples, computing device 76 may determine the size and position of the window according to changes in the beat (e.g., amplitude changes with respect to a baseline value), P, Q, R, or S points of a beat, changes in the derivative of the signal associated with the beat, or maximum or minimum points of a beat. Alternatively or additionally, computing device 76 may set an initial window around a characteristic of a beat (e.g., an R-wave point of the beat) and increase or decrease the size of the window according to characteristics of the samples included in the initial window. For example, if the samples of a current windowed beat are [1, 2, 3, 4, 5, 5, 4, 3, 2, 1], computing device 76 may use the history of the samples (e.g., samples decremented by one) to generate a wider window. Computing device 76 may expand the window to include [−4, −3, −2, −1, 0, 1, 2, 3, 4, 5, 5, 4, 3, 2, 1, 0, −1, −2, −3, −4]. In another example, computing system 76 may be configured to repeat the last known sample. For example, computing device 76 may expand the window to include [1, 1, 1, 1, 1, 1, 2, 3, 4, 5, 5, 4, 3, 2, 1, 1, 1, 1, 1, 1].

Applying a customizable window may help to ensure, for example, that only a single beat is being captured at one time. Capturing more than one beat with a window may lead to inaccurate comparison results, as the template beats are typically singular beats.

After a beat has been captured, computing device 76 correlates the windowed beat to one or more predetermined beat templates (282). The beat templates may represent known beat patterns and may be stored, for example, in a template database. According to some aspects of the disclosure, computing device 76 correlates the windowed beat to a template beat by generating a normalized cross correlation value between two beats. In some examples, computing device 76 generates a single cross correlation value for the windowed beat and the template beat. In other examples, computing device 76 generates multiple correlation values for the windowed beat and the template beat by “sliding” the template beat in multiple positions with respect to the windowed beat, and generating a correlation value at each position. In other examples, computing device generates multiple correlation values for the windowed beat and the template beat by positioning the template beat in a plurality of specific positions with respect to the windowed beat, and generating a correlation value at each specific position. In other examples, computing device 76 correlates the beats by generating a profile of the windowed beat according to certain characteristics of the beat, and comparing the profile of the windowed beat to a profile of a template beat.

In some examples, computing device 76 correlates the windowed beat to a template beat by generating a single normalized cross correlation value between the windowed beat and the template beat. For example, computing device 76 may compare all of the samples associated with a windowed beat to all of the samples associated with the template beat to generate a correlation value. In some examples, computing device generates the normalized cross correlation value using Equation (1) below:

$$ρ = \frac{1}{n - 1} {\sum }_{i = 1}^{n} \frac{( {x}_{i} - \overline{x} ) ( {y}_{i} - \overline{y} )}{{σ}_{x} {σ}_{y}} \qquad ( 1 )$$

where xᵢ represents a current sample of the windowed beat; y, represents a current sample of the template beat; x̅ represents a mean of windowed beat samples; y represents a mean value of template beat samples; σₓ represents a standard deviation of all windowed beat samples; and σ_{y} represents a standard deviation of all template beat samples.

Computing device 76 may, according to some examples, generate a single correlation value by aligning the windowed beat and the template beat according to a “best fit” position and calculating the normalized cross correlation value shown in Equation (1). For example, computing device 76 determines a peak point, marker point, or other characteristic of the template beat and aligns the peak point of the template beat with a peak point of the windowed beat. Computing device 76 then generates the normalized cross correlation value between the windowed beat and the template beat. The cross correlation may be, in some cases, indicative of a similarity between the windowed beat and the template beat.

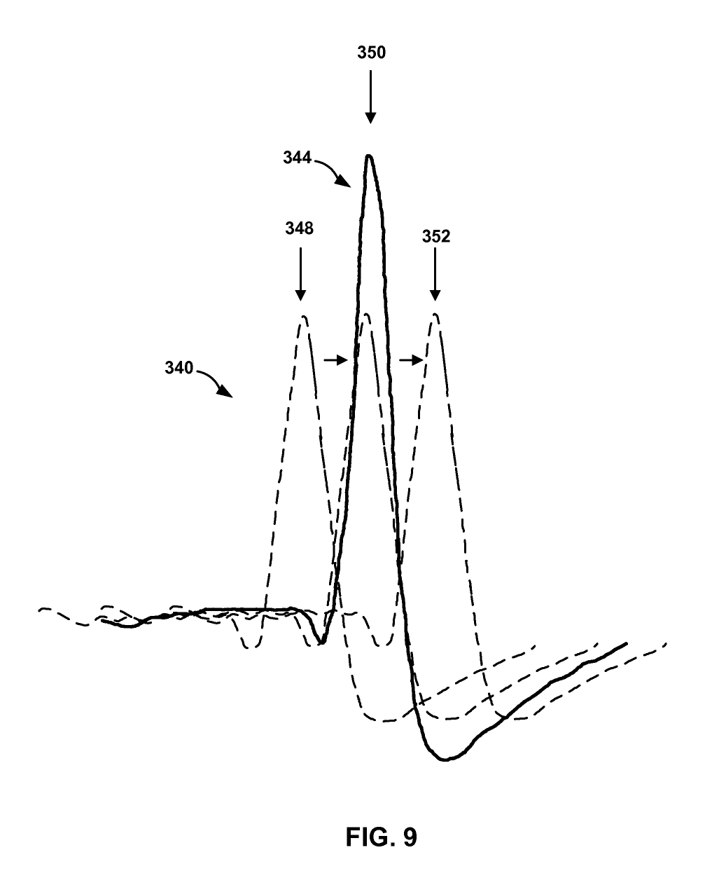

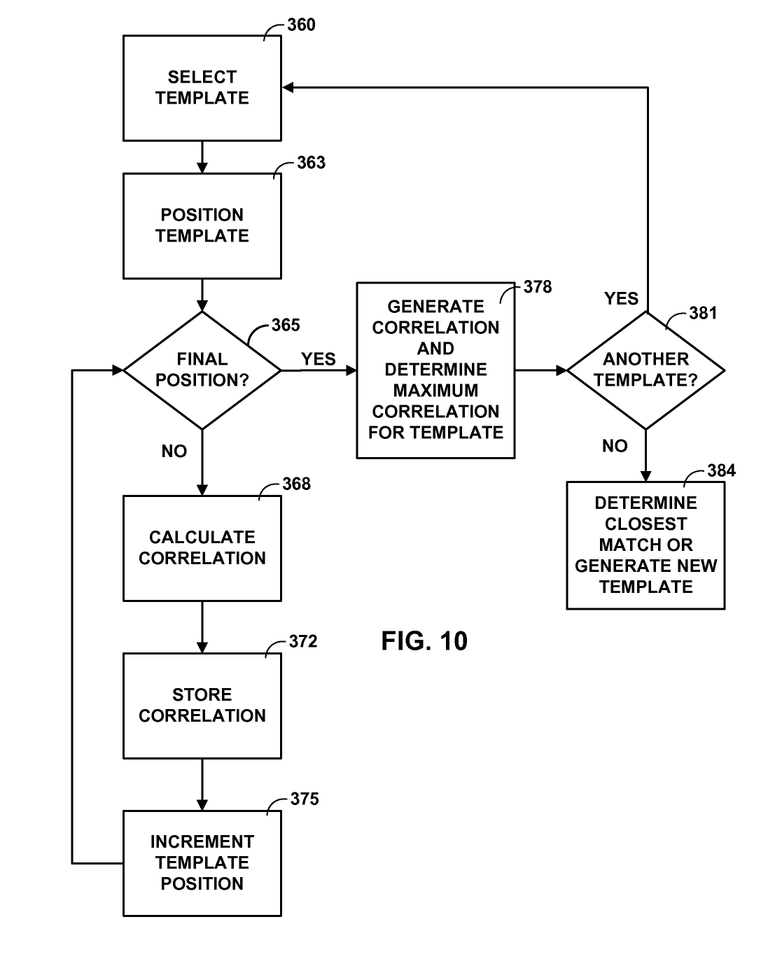

In other examples, computing device 76 may correlate a windowed beat to a template beat (282) by generating a plurality of correlation values for the same windowed beat and template pair. For example, as described in greater detail with respect to FIGS. 9 and 10, computing device 76 generates a plurality of correlation values by positioning or “sliding” the template beat over a beat under consideration, and generating a correlation value at each incremental position. Computing device 76 can then determine the maximum correlation between the windowed beat and the template beat by determining a maximum cross correlation value of all of the increments. The maximum correlation may then be accepted, by classifier module 216 of computing device 76, as the final correlation value between the template and the beat. Generating multiple cross correlations for the same windowed beat/template beat pair at different intervals may reduce correlation errors introduced by errors in finding a single, “best fit” position at which to apply the template.

In other examples, computing device 76 may correlate a windowed beat to a template beat (282) by generating a plurality of correlation values for the windowed beat and a template beat by positioning the template beat in a plurality of specific positions with respect to the windowed beat. Computing device 76 then generates a correlation value at each of the designated positions. For example, computing device 76 may align the beats according to the P, Q, R, and/or S points of the beats, according to the maximum or minimum values of the beats, or according to other beat characteristics, and generate a correlation value at each of the points. Computing device 76 can then determine the maximum correlation between the windowed beat and the template beat by determining a maximum cross correlation value of all of the positions. The maximum correlation may then be accepted, by classifier module 216 of computing device 76, as the final correlation value between the template and the beat.

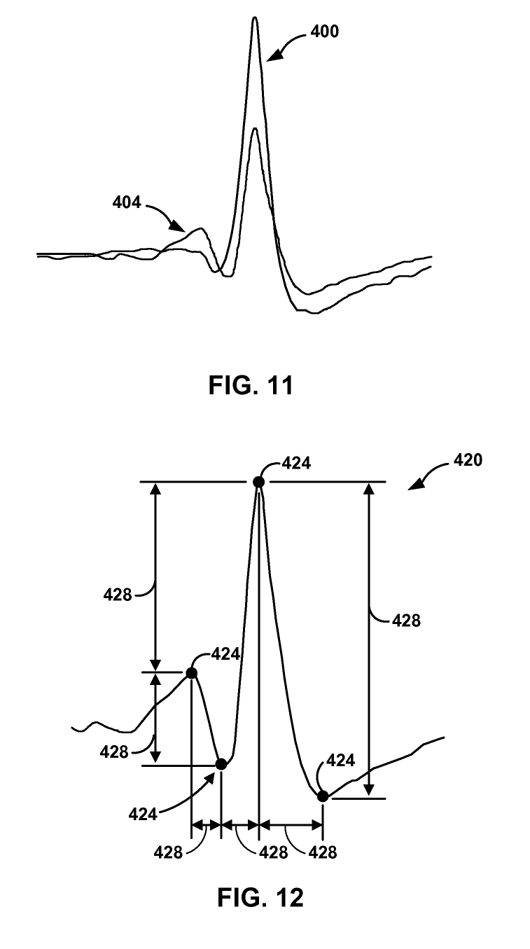

In other examples, computing device 76 can also correlate beats (282) in a variety of manners other than using generating cross correlation values (e.g., generating values using Equation (1)). For example, as described in greater detail with respect to FIGS. 11-13, computing device 76 may also correlate beats by generating a profile of characteristics of a windowed beat, and comparing the profile of the windowed beat to a template beat having known characteristics. Example characteristics may include an amplitude of inflection points of the windowed beat, width between inflection points of the windowed beat, or other beat characteristics. For example, other characteristics may include one or more slope measurements of the beat, as well as notching associated with the beat (e.g., a “notched” portion of a wave wherein the amplitude of the wave decreases slightly and subsequently increases, thereby creating a depression, or notch, in the wave).

In some examples, computing device 76 may assign more weight to some characteristics of a given profile when comparing the profile of a windowed beat to a profile of a template beat. For example, computing device 76 may generate a profile correlation score that is made up of correlation values for each characteristic included in the profiles. In such an example, computing device 76 may give more weight to the amplitude measurements of the profile than to width measurements between inflection points of the profile or to the slope measurements of the profile. Accordingly, computing device 76 may indicate a high correlation between a windowed beat and a template beat if the amplitudes of the beats are similar, even if other characteristics of the profiles are not as highly correlated. In some examples, computing device 76 can dynamically change the weights assigned to certain characteristics of the profile according to characteristics of the EGM signal. For example, computing device 76 may alter a weight assigned to the amplitude characteristics of a beat based on the resolution of the EGM signal (e.g., a low resolution EGM signal causes computing device 76 to assign a higher weight to the amplitude characteristics).

According to some aspects of the disclosure, computing device 76 may implement more than one method of correlating beats (282) simultaneously or in succession. For example, computing device 76 may complete a plurality of correlation methods for a single windowed beat. Computing device 76 may independently evaluate each of the implemented correlation methods or weigh the results of the correlation methods according to a predetermined algorithm (e.g., provide a different importance, or weight, to each correlation method of a plurality of correlation methods). The amount of importance assigned to a certain correlation method may be determined through testing. For example, a trained professional may determine the optimal weights for each correlation method of a multiple correlation method system by adjusting different weighting scenarios using a known EGM dataset. The trained professional can select the optimal weights to be applied to the correlation methods to achieve the most accurate comparison between a windowed beat and a template beat. For example, the trained professional can visually indentify when beats are similar, and select the weighting scheme that produces a high correlation value for similar beats. Further, the trained profession can visually identify when beats are not similar, and select a weighting scheme that ensures a high correlation value is not produced. In some examples, the weighting scheme may also be altered during processing. For example, a user, such as a trained professional, can monitor the accuracy of the weighting scheme and periodically provide input to optimize the weighting scheme.

According to an example in which multiple correlation methods are implemented, computing device 76 may first determine a correlation between a windowed beat and a template beat by “sliding” the template beat over the windowed beat incrementally, calculating a cross correlation at each increment, and selecting a maximum correlation between the windowed beat and the template beat (e.g., FIGS. 9-10). Computing device 76 may then verify those correlation results by generating a profile of characteristics of the windowed beat, and comparing the profile of the windowed beat to a profile of a template beat having known characteristics (e.g., FIGS. 11-13). In some examples, computing device 76 may assign higher weight to the results of the profile correlation. For example, computing device 76 may determine that the windowed beat and the template beat are not highly correlated, even if the beats have a relatively high cross correlation value if the beat profiles are not highly correlated.

In other examples, computing device 76 may also dynamically alter the manner in which multiple correlation methods are used. For example, computing device 76 may dynamically change the weighting assigned to a correlation value generated using a correlation equation and a profile correlation according to the resolution of the EGM signal. In such an example, computing device 76 may assign relatively less weight to a cross-correlation calculation and relatively more weight to a profile correlation for an EGM signal having a relatively low resolution.

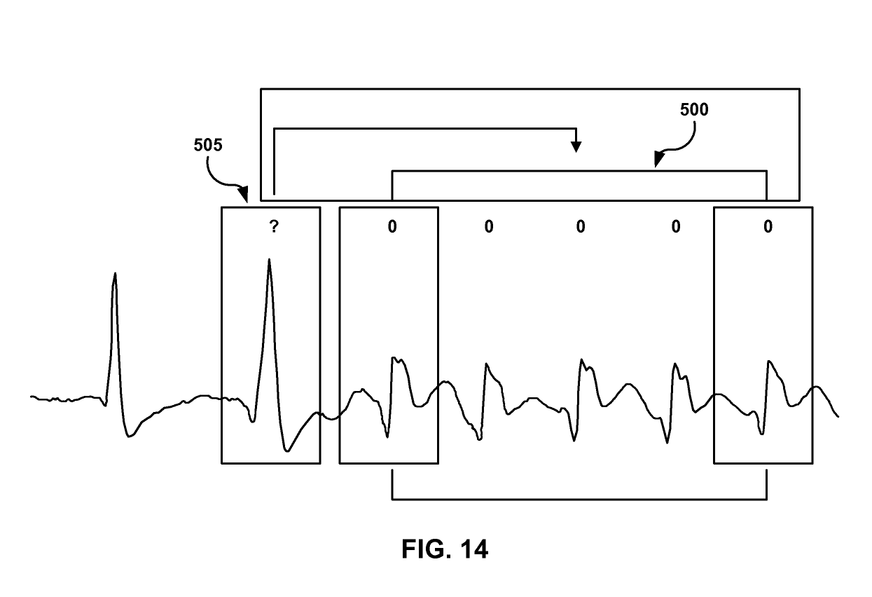

Referring still to FIG. 6, after correlating beats (282), computing device 76 can group similarly correlated beats of an EGM together (284). For example, according to some aspects of the disclosure, computing device 76 assigns each analyzed beat to a template according to the correlation results of step 282. Computing device 76 then groups the beats according to the template assignment. Grouping similarly correlated beats of an EGM may allow computing device 76 to identify changes in morphology. For example, computing device 76 may recognize a change in morphology based on a transition one group of beats to another group of beats in successive beats. In addition, computing device 76 can update or modify templates based on new beats being assigned to a group. For example, as described in greater detail with respect to FIGS. 14-17, computing device 76 may group beats (284) and generate new templates from the groups of beats in a variety of manners. In some examples, computing device 76 generates a template by averaging all of the beats of a particular group. In other examples, computing device 76 generates more than one template by selecting a plurality of beats of a group of beats as representative beats.

Grouping beats may help to prevent false identifications of morphology changes (“false positives”). For example, beats of an EGM may change slightly over time. Slight changes in beat shape may be exaggerated if computing device 76 is comparing two beats that are not close in time. As such, if computing device 76 generates if a template beat on the first beat of an EGM and applies that template to all of the beats of the EGM, a beat later in time may not closely correlate with the template beat, even though the beat is the same type of beat as the template beat. Grouping beats and generating templates according to the groups can smooth inherent inconsistencies in beats over time.

Computing device 286 also makes a morphology change decision based on beat correlations and/or beat groupings (286). For example, computing device determines when morphologies of a given EGM change based on beat correlations and/or beat groupings (286). In some examples, computing device 76 uses detected changes in morphology to classify an episode (e.g., classify a VT, VF, or SVT episode). In other examples, computing device 76 uses detected changes in morphology to verify identifications of episodes by an IMD, such as IMD 16.

FIG. 7 is a representation of an EGM 300 having an analysis window 304 that has been applied to a single beat 308 having a plurality of associated profile points 312. In some examples, the processes described herein as being carried out on EGM 300 may be carried out by computing device 76 (FIGS. 2 and 4). For example, computing device 76 may apply analysis window 304 and profile points 312 to beat 308 of EGM 300 using classifier module 216. Accordingly, for purposes of illustration only, the method of FIG. 6 is described with respect to computing device 76 shown in FIG. 4, though various other systems and/or devices may be utilized to implement or perform the EGM processing shown in FIG. 7.

Computing device 76 may apply window 304 around beat 308 of EGM 300 in order to separate beat 308 from the other beats of EGM 300. In some examples, computing device 76 applies window 304 beat 308 of EGM 300 before comparing beat 308 to one or more template beats (e.g., digital samples of a known heartbeat waveform that can be used as a reference for comparison with other beats). For example, window 304 helps to isolate beat 308 for comparison to a single template beat. Isolating beat 308 can help to reduce possible inaccuracies associated with comparing more than one current beat (or only a portion of a current beat) to a template beat.

In some examples, as described in greater detail with respect to FIG. 8 below, computing device 76 identifies a certain number of profile points 312 associated with beat 308 prior to setting the width and position of window 304. Profile points 312 may be identified, for example, based on inflection points of the beat, the amplitude of the beat at P, Q, R, or S points, changes in the derivative of the beat signal, interval between maximum or minimum amplitude points of a beat, points at which the signal of the beat crosses an x-axis, or other profile points. After the profile points 312 have been identified, computing device 76 can accurately determine the beginning and ending points of beat 308 and apply window 304 according to those beginning and ending points. For example, after identifying profile points 312, computing device 76 sets window 304 wide enough to capture all of the profile points 312 of beat 308.

FIG. 8 is a flow diagram illustrating an example method for applying an analysis window to a heartbeat of an EGM. In some examples, the method shown in FIG. 8 may be carried out by computing device 76. For example, the method shown in FIG. 8 may be carried out by episode classifier module 216 to detect changes in morphology. Accordingly, for purposes of illustration only, the method of FIG. 8 is described with respect to computing device 76 shown in FIG. 4 and EGM 300 of FIG. 7, though various other systems and/or devices may be utilized to implement or perform the method shown in FIG. 8. For example, in some other embodiments, the method shown in FIG. 8 may be carried out by programmer 64 (FIG. 2) or IMD 16 (FIG. 1).

In some examples, computing device 76 initializes analysis of an EGM signal, such as EGM 300 shown in FIG. 7 (320). Computing device 76 may initialize analysis of EGM 300 to detect changes in morphology. After analysis has begun, computing device 76 evaluates EGM 300 to identify one or more characteristics the signal. For example, computing device 76 may identify changes in EGM 300 with respect to a baseline, a positive or negative slew (e.g., events in the derivative of the EGM signal), maximum or minimum points of the signal, etc. After collecting one or more characteristics of EGM 300, computing device 76 determines the end points of a single beat, such as beat 308 (324). For example, computing device 76 utilizes the gathered characteristics to determine a starting point and an ending point for a particular beat. Computing device 76 then sets a window, such as window 304, around the starting and ending points of analysis to set a beat 308 apart from other beats of EGM 300 (326).

The method shown in FIG. 8 can be repeated for each beat of EGM 300. Because the window of analysis is tailored to the characteristics of a beat, the window is customizable for each beat under analysis. Applying a customizable window may aid in capturing a single beat at a time (e.g., rather than capturing a partial beat, or capturing more than one beat).

FIG. 9 is a representation of correlating a template heartbeat 340 to a heartbeat under analysis 344 at multiple positions. In some examples, the correlation described in connection to FIG. 9 may be carried out by computing device 76 (FIGS. 2 and 4). For example, computing device 76 may correlate a template beat to a beat under analysis at one or more positions using classifier module 216. Accordingly, for purposes of illustration only, FIG. 9 is described with respect to computing device 76 shown in FIG. 4, though various other systems and/or devices may be utilized.

FIG. 9 shows template beat 340 being positioned in time relative to beat 344 under analysis at a first position 348, a second position 350, and a third position 352 in time, where time proceeds from left to right. In some examples, the disclosure the distance between positions 348-352 may be more or less than those shown in FIG. 9, and additional, other positions may also be included. In the example shown in FIG. 9, computing device 76 generates a correlation value between template beat 340 and beat 344 when template beat 240 is placed at each position 348-352. For example, computing device 76 may generate a correlation value between template beat 340 and beat under analysis 344 using Equation (1) above, although other methods of determining a correlation value could also be implemented. Computing device 76 can then use the correlation value to determine how similar beat under analysis 344 is to template beat 340. The positioning of template beat 340 to obtain a maximum correlation may be important in determining an actual correlation between template beat 340 and beat under analysis 344. For example, beats that are not aligned properly may result in a low correlation value, even though the template beat and beat under analysis are similar.

While FIG. 9 shows template beat 340 in three positions 348-352, computing device 76 may position template beat 340 in fewer or many more positions than those shown in FIG. 9. In some examples, computing device 76 incrementally “slides” template beat 340 over beat under analysis 344, e.g., in a series of fixed size or differently sized increments, and generates a correlation value at each increment. Computing device 76 may implement 3, 5, 10, 15, 50, or any other number of incremental positions for application of template beat 340 relative to beat 344 in order to determine a correlation between template beat 340 and beat under analysis 344. The maximum correlation value produced among the positions is then accepted as the final correlation value between the template beat 340 and beat 344.

FIG. 10 is a flow diagram illustrating an example method of positioning a template heartbeat in a plurality of positions with respect to a beat under analysis, calculating correlation values at each position, and determining a position in which the correlation between the template beat and the beat under analysis is at a maximum (e.g., a maximum correlation value). In some examples, the method shown in FIG. 10 may be carried out by computing device 76. For example, the method shown in FIG. 10 may be carried out by episode classifier module 216 to detect changes in morphology. Accordingly, for purposes of illustration only, the method of FIG. 10 is described with respect to computing device 76 shown in FIG. 4, though various other systems and/or devices may be utilized to implement or perform the method shown in FIG. 10. For example, in some other embodiments, the method shown in FIG. 10 may be carried out by programmer 64 (FIG. 2) or IMD 16 (FIG. 1).

In some examples, computing device 76 initially selects a template, such as template beat 340 shown in FIG. 8, to compare to a beat under analysis, such as beat 344 (236). Computing device 76 may select the last beat under analysis as a template, or may select a template from a database of known templates. In other examples, as described in greater detail with respect to FIGS. 14-17, computing device 76 may generate and select templates in a variety of other ways.

After selecting template beat 340 (360), computing device 76 positions template beat 340 relative to beat under analysis 344 (363). For example, computing device 76 may attempt to align template beat 340 and the beat under analysis 344 according to peak amplitudes of template beat 340 and beat under analysis 344 (e.g., by aligning the peak of the R-waves of template beat 340 and beat under analysis 344). Computing device 76 may then reposition template beat 340 with respect to the first position (e.g., by incrementally moving template beat 340 left or right with respect to beat under analysis 344). In other examples, computing device 76 may position template beat 340 at a first position prior to incrementally sliding template beat 340 over the beat under analysis 344 from left to right. Computing device 76 then determines if the position of template beat 340 is the final position for generating a correlation value for the selected template (365). For example, in methods that calculate a correlation value at more than one position for a given template (e.g., a predetermined, “n” number of positions), computing device 76 determines whether the current position is the final position for determining a correlation value.

If the current position of the template is not the final position, computing device 76 generates a correlation value (368), stores the correlation value (372) and increment or decrement the position of template beat 340 in time (375). For example, computing device 76 may increment the position of template beat 340 by repositioning template beat 340 to the right of the last position, or decrement the position of template beat by repositioning template beat 340 to the left of the last position. Computing device 76 can then generate a correlation value for the next position of template beat 340 following steps 365-375. Computing device 76 may be configured to generate a correlation value in a variety of ways. For example, as described with respect Equation (1) above, computing device 76 may generate a cross correlation between the template beat and the beat under analysis. In another example, computing device 76 may generate a profile of the beat under analysis, and correlate the profile of the beat under analysis to the template (e.g., as described with respect to FIGS. 11-13).

Upon reaching the final position for the selected template, computing device 76 generates a final correlation value and determines a maximum correlation for the template (378). For example, computing device 76 may analyze all of the correlation values that were incrementally generated for different template positions and select the highest correlation between the template beat and the beat under analysis. In some examples, computing device 76 may then use the highest correlation value to determine whether the beat under analysis is similar to the template beat.

In some examples, multiple beat templates may be compared to a beat under analysis. In such examples, after determining a maximum correlation value for a particular beat template 340, computing device 76 may determine whether there are any other beat templates to compare to the beat under analysis (381). If there are other beat templates for comparison to the beat under analysis, computing device 76 may select the next beat template and begin the process shown in FIG. 10 again.

If there are no other beat templates to compare to the beat under analysis 344, computing device 76 determines the closest match beat template to the template under analysis, or generates a new beat template (384). For example, if the beat under analysis 344 is highly correlated to one of the beat templates, computing device 76 may determine that the beat under analysis should be labeled according to that template. If, however, none of the beat templates correlate with the beat under analysis 344, the beat under analysis may be used to generate a new beat template.

FIG. 11 is a representation of a first beat 400 and a second beat 404 that are positioned with their peaks in alignment. According to some metrics, beats 400 and 404 are not similar. For example, beat 404 has larger deviations from a baseline prior to its peak value, while beat 400 exhibits a lower minimum point than beat 404. Despite the differences between the beats, some correlation metrics may identify the beats as being similar. For example, according to some correlation calculations, the beats may be identified as being 95% similar. Accordingly, in some instances, another correlation metric can be used to determine a correlation between beats. For example, in some examples, comparing specific beat characteristics provides an additional metric with which to compare beats.

FIG. 12 is a representation of a heartbeat having an associated heartbeat profile 420.

In some examples, profile 420 may be generated by computing device 76 (FIGS. 2 and 4). For example, computing device 76 may identify data points, generate measurements, etc., using classifier module 216. Accordingly, for purposes of illustration only, FIG. 12 is described with respect to computing device 76 shown in FIG. 4, although various other systems and/or devices may be utilized.

In some examples, computing device 76 may generate profile 420 of a beat under analysis to compare to a known template profile generated from a template beat. The profile 420 may be compared to the template profile to determine how correlated the beat under analysis is to the template beat. Profile 420 may include a variety of beat characteristics and/or measurements. For example, computing device 76 may generate profile 420 in a variety of ways, identifying and measuring a variety of different beat characteristics. For example, computing device 76 may identify inflection points 424 of the beat, and generate a variety of measurements 428 associated with inflection points 424. Computing device 76 may calculate the distance in time between inflection points 424, amplitude changes between inflection points 424, or other measurements associated with inflection points 424. Computing device 76 may also identify or calculate a variety other beat characteristics for profile 420. For example, computing device 76 may also identify “notching” in the beat under analysis, and include the notching metric in profile 420.

According to one aspect of the disclosure, computing device 76 generates a profile by first identifying an initial starting point and an ending point for a beat. In some examples, the starting point and ending point for the beat are chosen according to the window that is applied to the beat (as described, for example, according to FIGS. 7 and 8). The starting and ending points are chosen far enough in time from each other that an entire beat is captured, but close enough in time so that portions of adjacent beats are not captured. The starting and ending points, e.g., size of the window, may vary based on heart rate. In the case of an R-wave, computing device 76 then builds the profile by identifying a maximum point (e.g., the maximum voltage of the EGM signal) as an R-point, and two most minimum points (e.g., the two most minimum voltages of the EGM signal) as a Q-point and an S-point. Computing device 76 then identifies whether notching is present in the R-wave, for example, by determining whether inflection points are present in the R-wave. After determining the Q, R, and S points of the wave, computing device 76 identifies local maxima to each side (e.g., forward and backward in time) of the identified Q and S points. Computing device 76 identifies the local maxima as a P-point and a T-point. The resulting profile includes, then, the dimensions of certain beat features (e.g., amplitudes of Q, R, S, P, and T waves) as well as the total duration of the beat, and whether notching is present in the R-wave.

Profile 420 may include certain correlation metrics that cannot be evaluated using a correlation equation. For example, applying a profile analysis to the beats 400 and 404 shown in FIG. 11 may provide a more accurate indication of the correlation between beats 400 and 404 than applying a correlation equation. Accordingly, computing device 76 may compare each characteristic of a profile to a corresponding characteristic in a template profile. In some examples, the profile comparison is assigned a score based on how the characteristics in profile 420 compare to characteristics of a template profile. In some examples, certain characteristics may be weighted more heavily than others. For example, computing device 76 may assign more weight to some characteristics of profile 420 when comparing profile 420 to a template profile. In such an example, computing device 76 may give more weight to amplitude measurements of profile 420 than to duration measurements between inflection points of profile 420, or to slope measurements of profile 420. In some examples, computing device 76 can dynamically change the weights assigned to certain characteristics of profile 420 according to characteristics of the EGM signal. For example, computing device 76 may alter a weight assigned to the amplitude characteristics of profile 420 based on the resolution of the EGM signal (e.g., a low resolution EGM signal causes computing device 76 to assign a higher weight to amplitude characteristics).

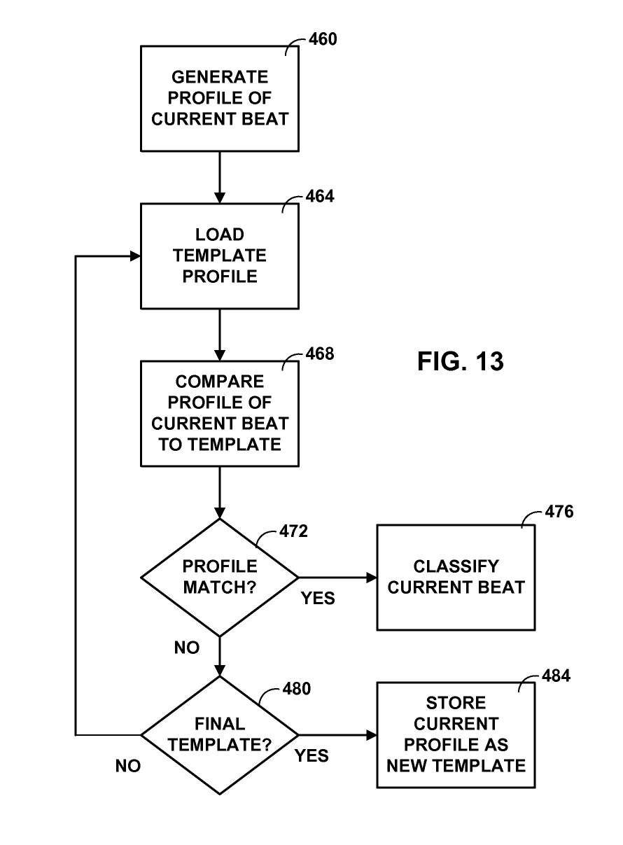

FIG. 13 is a flow diagram illustrating an example method of generating a heartbeat profile and applying the heartbeat profile to a number of heartbeat templates. In some examples, the method shown in FIG. 13 may be carried out by computing device 76. For example, the method shown in FIG. 13 may be carried out by episode classifier module 216 to detect changes in morphology. Accordingly, for purposes of illustration only, the method of FIG. 13 is described with respect to computing device 76 shown in FIG. 4, though various other systems and/or devices may be utilized to implement or perform the method shown in FIG. 13. For example, in some other examples, the method shown in FIG. 13 may be carried out by programmer 64 (FIG. 2) or IMD 16 (FIG. 1).

Computing device 76 first generates a profile, such as profile 420 shown in FIG. 12, of a beat currently being analyzed (460). As described with respect to FIG. 12, profile 420 may include a variety of characteristics of the beat under analysis. For example, profile 420 may include measurements 428 such as the distance between inflection points of the beat under analysis, the amplitude change between inflection points 424 of the beat under analysis, or other measurements associated with inflection points 424 of the beat under analysis. Profile 420 can be used to compare characteristics of the beat under analysis to a number of other template beats, and may include any useful measurement for comparison purposes.

After generating profile 420 of the beat under analysis (460), computing device 76 loads a template profile to compare to the profile of the beat under analysis (464). The template may be loaded from a data base of template profiles, or may be generated from the previous beat under analysis. Computing device 76 then compares profile 420 of the current beat under analysis to the template profile (468). Computing device 76 can use the comparison to determine a correlation between the beat under analysis and the template beat (472). For example, if the difference between profile 420 of the beat under analysis and the template profile is small, computing device 76 may determine that the profiles match. If, however, computing device 76 identifies differences between the profile of the beat under analysis and the template profile, computing device 76 may determine that the profiles do not match.

Computing device 76 may utilize one or more threshold values to determine whether the profiles match. For example, computing device 76 may determine that the profiles of the beat under analysis and the template beat do not match if the differences between one or more metrics associated with the profile (e.g., a distance between a pair of inflection points) exceed a predetermined value. Other methods of determining whether the profiles sufficiently match could also be used.

In some examples, computing device 76 may generate a single weighted profile score to determine whether profiles match. For example, computing device 76 may assign each comparison of characteristics in a profile a weight, and generate a composite score that accounts for each characteristic in a beat profile. In one example, computing device 76 generates a single weighted profile score according to Equation (2) below:

ρ=(w₁·c₁)+(w₂·c₂)+(w₃·c₃) (2)

where ρ is the correlation score, wₙ is a weight value, and cₙ is a comparison of a characteristic of a beat under analysis to a characteristic of a template beat (e.g., a comparison of amplitude values). Computing device 76 can use the weighted score to determine whether a beat under analysis is similar to a template beat. Computing device 76 may also use the weighted score to determine whether the beat under analysis can be grouped with other beats of the EGM. For example, if the beat under analysis has a weighted score that is similar to other, previously analyzed beats, computing device 76 may determine that the beat under analysis should be grouped with the previously analyzed beats. As described with respect to FIGS. 14-17, grouping beats may aid computing device 76 in identifying changes in morphology.

Additionally or alternatively, computing device 76 may generate multiple scores for each beat under analysis profile/template profile pair. For example, computing device 76 may, assign a likeness value to each comparison of characteristics included in a profile (e.g., a likeness value of “1” may be assigned to a characteristics of a template beat and a characteristic of a beat under consideration that are comparatively alike, and a likeness value of “0” for characteristics that are not alike). In this way computing device 76 can generate an array of likeness values. For example, for a profile that includes five characteristics, an array of likeness values may be represented as “0 1 1 0 1 1.” Computing device 76 can combine the array of likeness values to generate a single score (e.g., sum the likeness values, equaling four in the example provided above) to determine whether the beat under analysis is similar to the template beat. For example, a high aggregate score may be indicative of a high correlation between the beats. Computing device 76 may also use the comparison array to determine whether the beat under analysis can be grouped with other beats of the EGM. For example, if the beat under analysis has an array that is similar to other, previously analyzed beats, computing device 76 may determine that the beat under analysis should be grouped with the previously analyzed beats. As described with respect to FIGS. 14-17, grouping beats may aid computing device 76 in identifying changes in morphology.

If profile 420 of the beat under analysis matches the template profile, computing device 76 may classify or label the beat under analysis as being similar to the template profile. If profile 420 of the beat under analysis and the template profile do not match, computing device 76 may look for other template profiles to compare to the profile of the beat under analysis (480). For example, other template profiles may be available for comparison to the profile of the beat under analysis. If other template profiles are available, computing device 76 may return to step 464 and load the next template profile. In some examples, if computing device 76 has exhausted all template profiles, computing device 76 may store profile 420 of the beat under analysis as a new template (484) for future use.

In some examples, the method shown in FIG. 13 may be incorporated into a more extensive correlation algorithm. For example, the method shown in FIG. 13 may be a portion of a correlation algorithm that includes other correlation measurements, such as the cross correlation Equation (1). According to an aspect of the present disclosure, computing device 76 first determines a correlation between a beat under analysis and a template beat by “sliding” the template beat to multiple reference positions over the beat under analysis incrementally, calculates a cross correlation value at each increment, and selects a maximum correlation value between the beat under consideration and the template beat. Computing device 76 then verifies that the beat under analysis and the template beat are correlated by generating a profile of characteristics of the beat under analysis, and comparing the profile of the beat under analysis to a template beat having known characteristics.

Additionally or alternatively, a number of other correlations methods could also be implemented with the profile comparison method shown in FIG. 13. For example, computing device 76 may also determine a correlation between the beat under analysis and the template beat using Pearson's Correlation Coefficient, Lin's Concordance Correlation Coefficient, a Winsorized Correlation, or an outlier trimmed correlation.

Computing device 76 may independently evaluate each of the implemented correlation methods or weigh the results of the correlation methods according to a predetermined algorithm (e.g., provide a different importance, or weight, to each correlation method of a plurality of correlation methods). The amount of importance assigned to a certain correlation method may be determined through testing. For example, a trained professional may determine the optimal weights for each correlation method of a multiple correlation method system by adjusting different weighting scenarios using a known EGM dataset. The trained professional can select the optimal weights to be applied to the correlation methods to achieve the most accurate comparison between a beat under comparison and a template beat. For example, the trained professional can visually indentify when beats are similar, and select the weighting scheme that produces a high correlation value for similar beats. Further, the trained profession can visually identify when beats are not similar, and select a weighting scheme that ensures a high correlation value is not produced. In some examples, the weighting scheme may also be altered during processing. For example, a user, such as a trained professional, can monitor the accuracy of the weighting scheme and periodically provide input to optimize the weighting scheme.

FIG. 14 is a representation of an EGM having a group of similar heartbeats 500 that are averaged together and compared to a current beat under analysis 505. In some examples, beats 500 may be averaged and compared to beat 505 by computing device 76 (FIGS. 2 and 4). For example, computing device 76 may identify a group of beats, average the beats, and compare the average to a beat under analysis using classifier module 216. The beats may be averaged, for example, by mathematically averaging voltage values of the EGM signal for the group of beats. Accordingly, for purposes of illustration only, FIG. 14 is described with respect to computing device 76 shown in FIG. 4, though various other systems and/or devices may be utilized.

As described above, grouping beats may help to prevent false identifications of morphology changes (“false positives”). For example, beats of an EGM may change slightly over time. Slight changes in beat shape may be exaggerated if computing device 76 compares two beats that are not close in time. As such, if computing device 76 generates a template beat based on the first beat of an EGM and applies that template to all of the beats of the EGM, a beat later in time may not closely correlate with the template beat, even though the beat is the same type of beat as the template beat. Grouping beats and generating templates according to the groups can smooth inherent inconsistencies in beats over time.

In some examples, computing device 76 assigns a beat under analysis to a group of beats according to a template that exhibits the highest correlation to the beat under analysis. For example, computing device 76 may generate a template (e.g., “Template 0”) for the first beat of an EGM, or select a template beat from a number of stored template beats for the first beat of an EGM based on a correlation between the first beat and the templates. Computing device 76 may then assign subsequent beats that are highly correlated to Template 0 to that template group (e.g., beats determined to be highly correlated according to any of the correlation methods described above).

In some examples, computing device 76 gathers all of the beats of a group, and the beats to produce a single beat template that incorporates characteristics of all of the constituent beats of the group. The new group beat template can then be compared to other beats for purposes of determining changes in morphology. For example, if a current beat under analysis is not sufficiently similar to the group template beat, computing device 76 may identify a change in morphology.

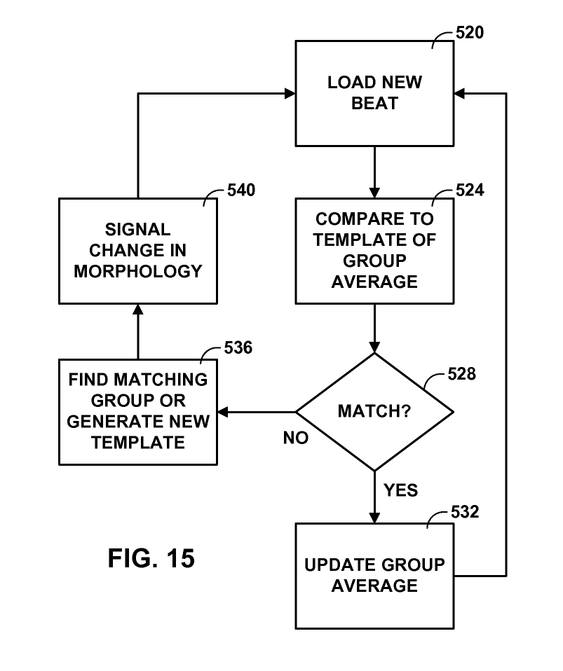

FIG. 15 is a flow diagram illustrating an example method of comparing a heartbeat to a heartbeat template generated from an average of a plurality of similar heartbeats. In some examples, the method shown in FIG. 15 may be carried out by computing device 76. For example, the method shown in FIG. 15 may be carried out by episode classifier module 216 to detect changes in morphology. Accordingly, for purposes of illustration only, the method of FIG. 15 is described with respect to computing device 76 shown in FIG. 4, though various other systems and/or devices may be utilized to implement or perform the method shown in FIG. 15. For example, in some other embodiments, the method shown in FIG. 15 may be carried out by programmer 64 (FIG. 2) or IMD 16 (FIG. 1).

In some examples, computing device 76 begins by loading a new beat (e.g., loading new digital samples corresponding to a beat) for analysis, such as beat 505 shown in FIG. 14 (520). Computing device 76 then compares beat under analysis 505 to a group average template beat (524). The group average template beat may be comprised of a number of similar beats. For example, as described above, computing device 76 may group beats based on the correlation of the beats to a template beat, such as group 500. Computing device 76 may generate a group average template beat by averaging a plurality of the beats belonging to a certain group. In some examples, the group average beat is comprised of all of the beats of group 500. In other examples, computing device may select certain beats of group 500, and generate a group average beat based on the selected beats from group 500.

Computing device 76 then determines whether beat under consideration 505 is correlated with the group average template beat (528). Computing device 76 may determine whether beat under analysis 505 is correlated to the group average template beat according to any of the correlation methods described herein. If beat under analysis 505 is sufficiently correlated to the group average template beat, computing device 76 can add beat under analysis 505 to group of beats 500 (532). Computing device can also update the group average template beat to include beat under analysis 505 (532).

In some examples, if beat under 505 analysis does not correlate well with the group average template beat, computing device 76 may compare beat 505 to other template beats to find a matching beat (536). In other examples, if computing device 76 does not find a suitable match with available stored template beats, computing device 76 may generate a new template beat (536). Computing device may also signal a change in morphology (540). For example, computing device 76 may notify a user (e.g., a trained professional, such as a clinician) that the morphology has changed when beat under analysis 505 does not match the group template beat.

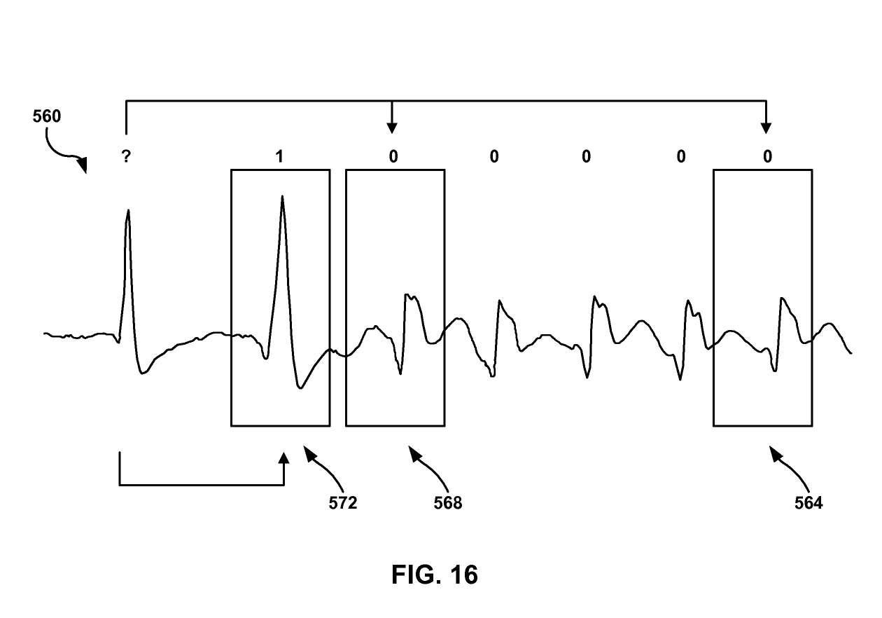

FIG. 16 is a representation of a heartbeat 560 of an EGM being compared a plurality of previous heartbeats. In some examples, beat 560 may be compared other previous beats by computing device 76 (FIGS. 2 and 4). For example, computing device 76 may identify a group of beats, select certain beats of the group, and compare the beats to a beat under consideration using classifier module 216. Accordingly, for purposes of illustration only, FIG. 16 is described with respect to computing device 76 shown in FIG. 4, though various other systems and/or devices may be utilized.

As described above, grouping beats may help to prevent false identifications of morphology changes. In some examples, computing device 76 assigns a beat under analysis to a group of beats according to a template that exhibits the highest correlation to beat under analysis 560. For example, computing device 76 may generate a template (e.g., “Template 0”) for the first beat of an EGM, or select a template beat from a number of stored template beats for the first beat of an EGM based on a correlation between the first beat and the templates. Computing device 76 may then assign subsequent beats that are highly correlated to Template 0 to that template group (e.g., beats determined to be highly correlated according to any of the correlation methods described above). In addition, upon identifying a beat that is not similar to a previously identified group, computing device 76 may assign the beat to a different template (e.g., “Template 1”). Accordingly, computing device 76 may assign a plurality of different templates to a plurality of different groups of beats of an EGM.

In some examples, computing device 76 may compare a beat under analysis, such as beat 560, to a beat selected from each identified group of beats. Computing device 76 may also compare beat under analysis 560 to more than one beat of a particular group. For example, in some examples, computing device 76 may compare beat 560 to a first and last beat of consecutive beats of a group. In other examples, computing device 76 may select other beats from a group of beats to compare to beat 560, or may compare all beats from a group of beats to compare to beat 560.

In the example shown in FIG. 16, computing device 76 compares beat under analysis 560 to a first beat 564 of a first group of beats (e.g., “Group 0”), as well as a last beat 568 of consecutive beats of the first group. Computing device 76 may also compare beat under analysis 560 to a beat 572 of a second group of beats (e.g., “Group 1”). In examples where Group 1 includes more than one beat, computing device 76 may compare beat under analysis 560 to multiple beats of Group 1. By comparing beat under analysis 560 to multiple beats, a the group having the highest correlation can be determined. Although the example of FIG. 16 shows the beats 564-572 as consecutive beats, in some examples, computing device 76 compares beat 560 to other beats that are not adjacent to beat 560. In addition, beats of a group, such as Group 0 shown in FIG. 16, need not be consecutive beats. For example, a group of beats may include similar beats that are spaced by one or more dissimilar beats.

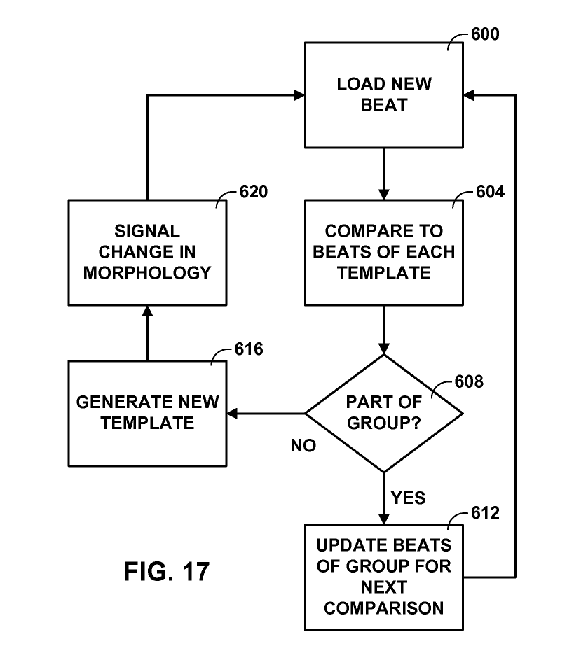

FIG. 17 is a flow diagram illustrating an example method of comparing a heartbeat to a plurality of heartbeat templates. In some examples, the method shown in FIG. 17 may be carried out by computing device 76. For example, the method shown in FIG. 17 may be carried out by episode classifier module 216 to detect changes in morphology. Accordingly, for purposes of illustration only, the method of FIG. 17 is described with respect to computing device 76 shown in FIG. 4, though various other systems and/or devices may be utilized to implement or perform the method shown in FIG. 17. For example, in some other embodiments, the method shown in FIG. 17 may be carried out by programmer 64 (FIG. 2) or IMD 16 (FIG. 1).

In some examples, computing device 76 begins by loading a new beat for analysis, such as beat 560 shown in FIG. 16 (600). Computing device 76 then compares beat under analysis 560 to one or more template beats of each group of beats that have been previously identified by computing device 76 (604). In some examples, computing device 76 may compare beat under analysis 560 to more than one beat of each group. For example, computing device 76 may compare beat under analysis 560 to a first beat of a particular group, such as beat 564 shown in FIG. 16, as well as the last beat of that group, such as beat 568 shown in FIG. 16. In other examples, computing device 76 may select any other beats from a group of beats to compare to beat 560.

Computing device 76 then determines whether beat under consideration 560 is correlated to with the selected template beat or beats (608). Computing device 76 may determine whether beat under analysis 560 is correlated to template beats according to any of the correlation methods described herein. If beat under analysis 560 is sufficiently correlated to the template beat or beats, computing device 76 can add beat under analysis 560 to the corresponding group of beats. Computing device 76 can also update the group of beats (612). For example, by adding the current beat under analysis 560 to the group of beats, the next beat under analysis can be compared to the current beat under analysis 560. In examples where computing device 76 compares a beat under analysis to the first and last beats of a given group, the last beat of the group is updated with the newly added beat.

In some examples, if beat under analysis 560 does not correlate well with any of the template beats of any of the groups, computing device 76 may generate a new template beat (616). Computing device 76 may also signal a change in morphology (540). For example, computing device 76 may notify a user (e.g., a trained professional, such as a clinician) that the morphology has changed (620). Changes in morphology can be used, for example, to classify an episode (e.g., classify a VT, VF, or SVT episode), or verify past identifications of episodes.

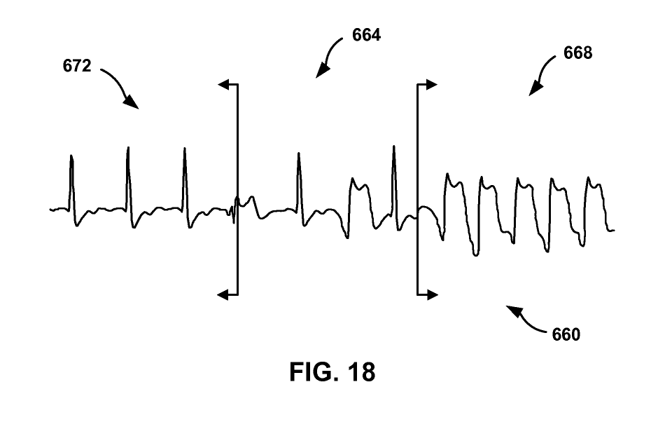

FIG. 18 is a representation of an EGM 600 having a transition period 664 between a first group of beats 668 and a second group of beats 672. In some examples, transition period 664 may be determined by computing device 76 (FIGS. 2 and 4). For example, computing device 76 may identify transition period 664 between beats 668 and beats 672 using classifier module 216. Accordingly, for purposes of illustration only, FIG. 18 is described with respect to computing device 76 shown in FIG. 4, though various other systems and/or devices may be utilized.

In some examples, computing device 76 identifies transition period 664 between first group of beats 668 and second group of beats 672. Identifying transition period 664 may aid in determining where a first predominant morphology ends and a second prominent morphology begins. By identifying transition period 664, computing device 76 more closely replicates a morphology change decision that may made by a trained professional, such as a clinician or electrophysiologist. For example, transition period 664 provides for a period of ectopy, so that computing device 76 does not identify several changes in morphology in short succession when there is truly only a single change in morphology.

Computing device 76 may remove certain beats of transition period 664 from consideration to avoid a false detection of multiple morphology changes. In some examples, computing device 76 determines transition period 664 by identifying a leftmost beat of beats belonging to a first morphology, such as beats 668, and a rightmost beat of beats belonging to a second morphology, such as beats 672. Computing device then sets the transition period to include all beats between the identified leftmost and rightmost beats, as these are the beats that the system identifies as being irregular (e.g., neither similar with the first morphology nor with the second morphology) in nature.

After identifying transition period 664, computing device 76 may then remove beats from consideration. For example, computing device determines which beat within transition period 664 has the fewest consecutive beats, and removes that beat from consideration. If no beat has more consecutive beats than another beat, both beats are removed consideration. Computing device 76 repeats the removal process until all beats of transition period 664 are similar. By recursively removing beats in the transition period, computing device 76 can better identify a transition from one morphology to another without falsely identifying multiple transitions.

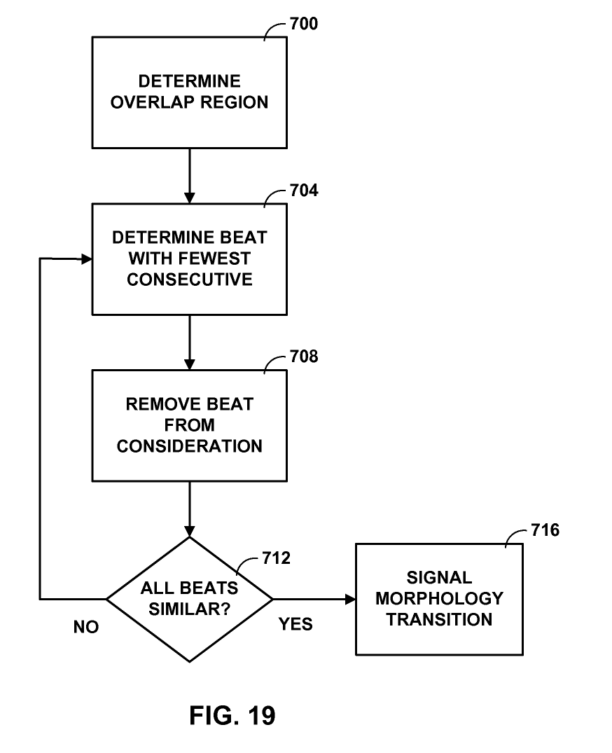

FIG. 19 is a flow diagram illustrating an example method of analyzing heartbeats associated with a transition period. In some examples, the method shown in FIG. 19 may be carried out by computing device 76. For example, the method shown in FIG. 19 may be carried out by episode classifier module 216 to detect changes in morphology. Accordingly, for purposes of illustration only, the method of FIG. 19 is described with respect to computing device 76 shown in FIG. 4, though various other systems and/or devices may be utilized to implement or perform the method shown in FIG. 19. For example, in some other embodiments, the method shown in FIG. 19 may be carried out by programmer 64 (FIG. 2) or IMD 16 (FIG. 1).

In some examples, computing device 76 determines an “overlap region,” such as transition period 664 shown in FIG. 18, between a first group of similar beats 668 and a second group of similar beats 672 (700). For example, transition region 664 may include interwoven beats from both first group 668 and second group 672, or other beats that are not shaped like the beats of first group 668 or second group 672. Transition period 664 may be selected by identifying a leftmost beat of first group of beats 668 and a rightmost beat of second group of beats 672. After identifying the transition period 664, computing device 76 determines which beat of transition period 664 has the fewest consecutive beats in transition period 664 (704). For example, transition period 664 may include a single beat from first group 668 and two consecutive beats from second group 672. In such an example, computing device 76 removes the single beat from consideration (708). Computing device 76 then determines whether all beats of transition period 664 are similar (712). After all of the beats of transition period 664 are similar, computing device 720 signals a change in morphology at the point of transition (716). If all of the beats of transition period 664 are not similar, computing device 76 returns to step 704 and repeats the process of determining which beat is the beat with the fewest consecutive beats.

Certain techniques of this disclosure are described as analyzing and processing electrogram (EGM) signals associated with an IMD configured to provide pacing functionality. However, in other examples, the techniques of the disclosure can be used to monitor, analyze, and process EGM signals, electrocardiogram (ECG) signals or other signals generated by other heart monitoring or pacing devices. For example, techniques of this disclosure may be implemented to analyze signals stored in an implantable loop recorder or other device. According to an aspect of the present disclosure, techniques described herein may be implanted to analyze signals stored by a Medtronic Reveal® insertable cardiac monitor.

The techniques described in this disclosure, including those attributed to image IMD 16, programmer 64, computing device 76 or various constituent components, may be implemented, at least in part, in hardware, software, firmware or any combination thereof. For example, various aspects of the techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components, embodied in programmers, such as physician or patient programmers, stimulators, image processing devices or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as random access memory (RAM), read-only memory (ROM), non-volatile random access memory (NVRAM), electrically erasable programmable read-only memory (EEPROM), FLASH memory, magnetic data storage media, optical data storage media, or the like. The instructions may be executed to support one or more aspects of the functionality described in this disclosure.

Various examples have been described. These and other aspects of the disclosure are within the scope of the following claims.

1. A method for processing a delivered cardiac signal, comprising:
receiving an electrogram signal that represents a plurality of heartbeats;
identifying a group of similar heartbeats of the plurality of heartbeats;
generating a template heartbeat based at least partially on the group of similar heartbeats, wherein the template heartbeat is generated by calculating an average of at least some of the group of heartbeats; and
comparing the template heartbeat to a heartbeat of the plurality of heartbeats that is not included in the group of similar heartbeats.

2. The method of claim 1, further comprising determining a change in morphology based at least partially on the comparison of the template heartbeat to the heartbeat of the plurality of heartbeats that is not included in the group of similar heartbeats.

3. The method of claim 1, further comprising separating the heartbeat that is not included in the group of similar heartbeats according to one or more characteristics of the heartbeat.

4. The method of claim 3, wherein separating the heartbeat that is not included in the group of similar heartbeats includes analyzing changes in the heartbeat with respect to a baseline, and separating the heartbeat from the group of heartbeats based at least partially on the changes in the heartbeat with respect to the baseline.

5. The method of claim 3, wherein separating the heartbeat that is not included in the group of similar heartbeats includes analyzing a slew associated with the heartbeat, and separating the heartbeat from the group of similar heartbeats based at least partially on the slew.

6. The method of claim 3, wherein separating the heartbeat that is not included in the group of similar heartbeats includes identifying a maximum point of the heartbeat and a minimum point of the heartbeat, and separating the heartbeat from the group of heartbeats based at least partially on the maximum point of the heartbeat and the minimum point of the heartbeat.

7. A method for processing a delivered cardiac signal, comprising:
receiving an electrogram signal that represents a plurality of heartbeats;
determining a group of similar heartbeats of the plurality of heartbeats;
generating a first template based on a first heartbeat of the group of similar heartbeats and a second template based on a second heartbeat of the group of similar heartbeats, wherein the second heartbeat is later in time than the first heartbeat; and
comparing the first template and the second template to a third heartbeat of the plurality of heartbeats.

8. The method of claim 7, wherein the first heartbeat is the first heartbeat identified as being included in the group of heartbeats and the second heartbeat is the last heartbeat identified as being included in the group of heartbeats.

9. The method of claim 7, further comprising identifying other heartbeats as belonging to the group of similar heartbeats and updating the second template with the other heartbeats as they are identified as belonging to the group of similar heartbeats.

10. The method of claim 7, further comprising identifying a plurality of groups of similar heartbeats, and generating a plurality of templates for each group of similar heartbeats of the plurality of groups of similar heartbeats.

11. The method of claim 7, further comprising separating the third heartbeat for comparing the third heartbeat to the first template and the second template according to one or more characteristics of the third heartbeat.

12. The method of claim 11, wherein separating the third heartbeat includes analyzing changes in the third heartbeat with respect to a baseline, and separating the third heartbeat based at least partially on the changes in the third heartbeat with respect to the baseline.

13. The method of claim 11, wherein separating the third heartbeat includes analyzing a slew associated with the third heartbeat, and separating the third heartbeat based at least partially on the slew.

14. The method of claim 11, wherein separating the third heartbeat includes identifying a maximum point of the third heartbeat and a minimum point of the third heartbeat, and separating the third based at least partially on the maximum point of the third heartbeat and the minimum point of the third heartbeat.

15. A method for processing a delivered cardiac signal, comprising:
receiving an electrogram signal that represents a plurality of heartbeats;
determining a first group of consecutive similar heartbeats of the plurality of heartbeats;
determining a second group of consecutive similar heartbeats of the plurality of heartbeats, wherein the second group of consecutive similar heartbeats is distinct from the first group of consecutive similar heartbeats; and
identifying a transition period between the first group of consecutive similar heartbeats and the second group of consecutive similar heartbeats, wherein the transition period includes one or more heartbeats that are not similar to the heartbeats of the first group or the heartbeats of the second group.

16. The method of claim 15, wherein the second group of heartbeats occurs later in time than the first group of heartbeats, and wherein identifying the transition period includes identifying a last heartbeat of heartbeats belonging to the first group of heartbeats, and identifying a first heartbeat of heartbeats belonging to the second group.

17. The method of claim 16, further comprising removing heartbeats from consideration within the transition period.

18. The method of claim 15, further comprising identifying a change in morphology based on the transition period.
